(19) United States
(12) Patent Application Publication
(10) Pub. No.: US 2019/0307735 A1
(43) Pub. Date: Oct. 10, 2019

(54) Composition and Methods for Treating Chronic Kidney Disease

(71) Applicant: MERCK SHARP & DOHME CORP, Rahway, NJ (US)

(72) Inventors: Jason M. Cox, East Windsor, NJ (US); Lijun Ma, Westfield, NJ (US); Xiaoyan Zhou, East Brunswick, NJ (US); Robin E. Haimbach, Pottstown, PA (US); Paul J. Coleman, Harleysville, PA (US); Haihong Zhou, Parlin, NJ (US); David E. Kelley, Westfield, NJ (US); Selwyn Aubrey Stoch, Livingston, NJ (US); Le T. Duong, Lansdale, PA (US); Maarten Hoek, South Orange, NJ (US)

(73) Assignee: MERCK SHARP & DOHME CORP, Rahway, NJ (US)

(21) Appl. No.: 16/444,843

(22) Filed: Jun. 18, 2019

Related U.S. Application Data
(63) Continuation of application No. 15/560,530, filed on Sep. 22, 2017.
(60) Provisional application No. 62/138,643, filed on Mar. 26, 2015.

(51) Int. Cl. A61K 31/444; A61K 31/55; A61K 31/4375; A61P 13/12; A61K 31/401; A61K 31/506; A61K 31/4178

(52) U.S. Cl. CPC A61K 31/444; A61K 31/55; A61K 31/4375; A61K 31/4178; A61K 31/401; A61K 31/506; A61P 13/12

(57) ABSTRACT

This invention relates to the treatment of chronic kidney disease, including diabetic nephropathy, focal segmental glomerulosclerosis (FSGS), nephrotic syndrome, non-diabetic chronic kidney disease, renal fibrosis or acute kidney injury by the administration of an RGD mimetic integrin receptor antagonist, either as a single agent or in combination with other agents.

### BACKGROUND OF THE INVENTION

The prevalence of chronic kidney disease (CKD) has reached epidemic proportions worldwide (Coresh J, Selvin E, Stevens L A et al. “Prevalence of chronic kidney disease in the United States,” JAMA, 2007; 298: 2038-47). In the US, more than 31 million patients live with chronic kidney disease, 40% due to diabetes. Diabetic nephropathy has been the leading cause of end-stage renal disease (ESRD) in the Western World. Chronic kidney disease includes diabetic nephropathy, focal segmental glomerulosclerosis (FSGS), nephrotic syndrome and non-diabetic chronic kidney disease.

Diabetic nephropathy is characterized by early podocyte injury, proteinuria, blood pressure elevation, a relentless decline in renal function and a high risk of cardiovascular disease. Focal segmental glomerulosclerosis (FSGS), which causes nephrotic syndrome, is another classic podocyte disease that progresses from podocyte injury to chronic kidney disease and end-stage renal disease (Fogo A B. “Causes and pathogenesis of focal segmental glomerulosclerosis,” Nat. Rev. Nephrol. 2014; Dec. 2).

The socio-economic impact of CKD and its complications are considerable. The annual cost of dialyzing diabetic patients in the US exceeds $17 billion. The current mainstay of treatment for patients with diabetic nephropathy and proteinuric chronic kidney disease (including FSGS) is renin-angiotensin system blockade. However, standard of care (hyperglycemia control and blockade of the angiotensin system) does not stop or reverse progression; thus additional renoprotective agents are needed for patients with diabetic nephropathy and proteinuric chronic kidney disease. See, Breyer M D. “Drug discovery for diabetic nephropathy: trying the leap from mouse to man,” Semin. Nephrol. 2012; 32(5): 445-51.

Proteinuria is a measure of glomerular barrier function and a hallmark of cardiovascular disease and most forms of chronic kidney disease. The glomerular podocyte plays a central role in the structural and functional integrity of the glomerular filtration barrier by extending microtubule-based major processes and actin-rich foot processes (FPs) around the underlying capillaries. See, Greka A, Mundel P. “Cell biology and pathology of podocytes,” Annu. Rev. Physiol. 2012; 74: 299-323. Dynamic actin cytoskeleton remodeling and attachment to glomerular basement membrane via integrins (α3ß1, αvß3) are pivotal to safeguard glomerular filter function.

Podocyte injury plays a key role in the initiation and progression of diabetic kidney disease (DKD). Multiple factors in diabetes cause abnormalities in podocyte signaling that lead to podocyte foot process effacement, hypertrophy, detachment, loss, and death. Numerous studies of human biopsy tissue have demonstrated a relationship between podocyte loss or pathological widening of podocyte foot processes and the albumin excretion rate (AER). Therefore, therapies aimed at limiting podocyte injury will have significant impact on novel treatment of patients with diabetic nephropathy.

Integrins are heterodimeric transmembrane glycoproteins that mediate cell-cell and cell-matrix interactions. Upon binding to the ligands in the extracellular matrix, integrins activate intracellular signaling and control various cell functions, including cell adhesion, proliferation, migration and ECM homeostasis. Based on their functions, Integrins are classified as collagen, laminin, and arginine-glycine-aspartic acid (RGD)-binding receptors, see, Pozzi A, Zent R. “Integrins in kidney disease,” J. Am. Soc. Nephrol. 2013; Jun. 24(7): 1034-9.

The αvß3 integrin belongs to the RGD-binding receptor class and modulates osteoclast function and angiogenesis. Compound A, a nonpeptide antagonist of αvß3 has been shown to increase bone density in postmenopausal women in a Phase II study. See, Nakamura I, Pilkington M F, Lakkakorpi P T, Lipfert L, Sims S M, Dixon S J, Rodan G A, Duong L T. “Role of alpha(v)beta(3) integrin in osteoclast migration and formation of the sealing zone,” J. Cell. Sci. 1999; November 112 (Pt 22):3985-93.; Perkins J J, Duong L T, Fernandez-Metzler C, Hartman G D, Kimmel D B, Leu C T, Lynch J J, Prueksaritanont T, Rodan G A, Rodan S B, Duggan M E, Meissner R S. “Non-peptide alpha(v)beta(3) antagonists: identification of potent, chain-shortened RGD mimetics that incorporate a central pyrrolidinone constraint,” Bioorg. Med. Chem. Lett. 2003 Dec. 15; 13(24):4285-8.; Murphy, M. G. et al. “Effect of L-000845704, an αvß3 integrin antagonist, on markers of bone turnover and bone mineral density in postmenopausal osteoporotic women,” J. Clin. Endocrinol. Metab. 2005; 90: 2022-2028.

Some αv integrins are expressed in the kidney and play important roles in development and progression of renal fibrosis. See, Ma L J, Yang H, Gaspert A, Carlesso G, Barty M M, Davidson J M, Sheppard D, Fogo A B. “Transforming growth factor-beta-dependent and -independent pathways of induction of tubulointerstitial fibrosis in beta6(−/−) mice,” Am. J. Pathol. 2003; 163(4): 1261-73. For example, αvß3 integrin mRNA expression was increased in the glomerular cells (including podocytes) of patients with diabetic nephropathy, see, Jin D K, Fish A J, Wayner E A, Mauer M, Setty S, Tsilibary E, Kim Y. “Distribution of integrin subunits in human diabetic kidneys,” J. Am. Soc. Nephrol. 1996; Dec. 7, (12): 2636-45. Published literature suggests that integrins (including αvβ3, αvβ1, αvβ6, α2β1) are expressed in the kidney and play important roles in modulation of glomerular filtration barrier and renal fibrosis; for example integrin αvβ3 plays a role in regulating the glomerular filtration barrier and may contribute to focal segmental glomerulosclerosis (FSGS), see, Pozzi A, Zent R. “Integrins in kidney disease,” J. Am. Soc. Nephrol. 2013; Jun. 24, (7): 1034-9.

Renal fibrosis is the hallmark of chronic kidney disease, regardless of underlying etiology. The pathological finding of renal fibrosis is characterized by progressive tissue scarring including glomerulosclerosis, tubulointerstitial fibrosis and loss of renal parenchyma (including tubular atrophy, loss of capillaries and podocytes). Several lines of evidence suggest that integrins play a role in the process of renal fibrosis. Deletion of αv-Integrin specifically in Pdgfrb cell subtypes led to protection against unilateral ureteral obstruction [UUO] inducted renal fibrosis suggesting that RGD integrins play an important in the development of renal fibrosis, see Henderson N C, Arnold T D, Katamura Y, Giacomini M M, Rodriguez J D, McCarty J H, Pellicoro A, Raschperger E, Betsholtz C, Ruminski P G, Griggs D W, Prinsen M J, Maher J J, Iredale J P, Lacy-Hulbert A, Adams R H, Sheppard D Nat Med. 2013 December; 19(12):1617-24.

Of the RGD integrins, the αvβ6 integrins have been shown to bind the LAP/TGF-β complex and activate TGFβ, see Munger JS1, Huang X, Kawakatsu H, Griffiths M J, Dalton S L, Wu J, Pittet J F, Kaminski N, Garat C, Matthay M A, Rifkin D B, Sheppard D. Cell. 1999 Feb. 5; 96(3):319-28. Genetic ablation of the β6 gene alleviates renal fibrosis in an Alport mice model. Furthermore, treatment of the Alport mice with anti-αvβ6 blocking mAbs led to inhibition of kidney fibrosis, see Hahm K I, Lukashev M E, Luo Y, Yang W J, Dolinski B M, Weinreb P H, Simon K J, Chun Wang L, Leone D R, Lobb R R, McCrann D J, Allaire N E, Horan G S, Fogo A, Kalluri R, Shield C F 3rd, Sheppard D, Gardner H A, Violette S M, Am J Pathol. 2007 January; 170(1):110-25. Lastly, αvβ6 deletion was shown to be protective against tubulointerstitial fibrosis induced by unilateral ureteral obstruction (UUO), see Li-Jun Ma, Haichun Yang, Ariana Gaspert, Gianluca Carlesso, Melissa M. Barty, Jeffrey M. Davidson, Dean Sheppard and Agnes B. Fogo American Journal of Pathology, Vol. 163, No. 4, October 2003.

The role of integrins in acute kidney injury have been reported as well. β1 integrins have shown to dramatically change their distribution during ischemic renal injury, and contribute to epithelial cell exfoliation and regeneration. The administration of a β1 antibody preserved renal function, ameliorated tubular epithelial injury, and reduced pro-inflammatory cytokines, see Ana Molina, Maria Ubeda, Maria M. Escribese, et al. J Am Soc Nephrol 16: 374-382, 2005 and Anna Zuk, Joseph V. Bonventre, Dennis Brown, et al. Am. J. Physiol. 275 (Cell Physiol. 44): C711-C731, 1998.

It has been demonstrated that a soluble form of urokinase plasminogen receptor (uPAR), namely suPAR, interacts with and activates integrin αvß3 in podocytes, leading to FSGS in humans, see, Wei C, El Hindi S, Li J, Fornoni A, Goes N, Sageshima J, Maiguel D, Karumanchi S A, Yap H K, Saleem M, Zhang Q, Nikolic B, Chaudhuri A, Daftarian P, Salido E, Torres A, Salifu M, Sarwal M M, Schaefer F, Morath C, Schwenger V, Zeier M, Gupta V, Roth D, Rastaldi M P, Burke G, Ruiz P, Reiser J. “Circulating urokinase receptor as a cause of focal segmental glomerulosclerosis,” Nat. Med. 2011; 17(8): 952-60. In a lipopolysaccharide-mediated albuminuria model in mice, activation of integrin αvß3 by the uPAR promoted podocyte migration and albuminuria, see, Wei C, Möller C C, Altintas M M, Li J, Schwarz K, Zacchigna S, Xie L, Henger A, Schmid H, Rastaldi M P, Cowan P, Kretzler M, Parrilla R, Bendayan M, Gupta V, Nikolic B, Kalluri R, Carmeliet P, Mundel P, Reiser J. “Modification of kidney barrier function by the urokinase receptor,” Nat. Med. 2008; Jan. 14 (1): 55-63. Angiopoietin-like 3 also induced podocyte F-actin rearrangement through integrin α(V)β₃/FAK/PI3K pathway-mediated Rac1 activation (see, Lin Y Rao J, Zha X L, Xu H. “Angiopoietin-like 3 induces podocyte F-actin rearrangement through integrin α(V)β₃/FAK/PI3K pathway-mediated Rac1 activation,” Biomed. Res. Int. 2013; 135608.

Additional animal studies have been conducted relating to the effect of integrins on renal function. Blocking β3 integrin activation (aa 592-712, CD61, BD Pharmingen) prevented LPS-induced proteinuria in mice, see, Wei C, Möller C C, Altintas M M, Li J, Schwarz K, Zacchigna S, Xie L, Henger A, Schmid H, Rastaldi M P, Cowan P, Kretzler M, Parrilla R, Bendayan M, Gupta V, Nikolic B, Kalluri R, Carmeliet P, Mundel P, Reiser J. “Modification of kidney barrier function by the urokinase receptor,” Nat. Med. 2008; Jan. 14(1): 55-63. Recently, it was reported that treatment with VPI-2690B, a humanized αvß3 antibody against c-loop, for 10 weeks reduced urinary albumin creatinine ratio (ACR) in ZDSD rats, a rodent DN model, see, Maile L A, Gollahon K A, Liu, J W, Xiaong Y, Murji A, Meli C, Shea M, Rehman A, Clemmons D. “VPI-2690B, a novel αvb3 integrin antibody, reduces hyperglycemia induced changes in renal function in a rat model of DN,” ADA poster, 2014. Blocking ligand occupancy of the αvß3 integrin by a F(ab)2 fragment of anti-c-loop of αvß3 antibody for 18 weeks attenuated proteinuria and early histologic changes of diabetic nephropathy in diabetic pigs, see, Maile L A, Busby W H, Gollahon K A, Flowers W, Garbacik N, Garbacik S, Stewart K, Nichols T, Bellinger D, Patel A, Dunbar P, Medlin M, Clemmons D. “Blocking Ligand Occupancy of the αvß3 Integrin Inhibits the Development of Nephropathy in Diabetic Pigs. Endocrinology,” 2014; December 155(12): 4665-75. Anti-c-loop of αvβ3 antibody treatment also inhibited the progression of albuminuria in STZ-induced diabetic rats, see, Maile L A, Gollahon K, Wai C, Dunbar P, Busby W, Clemmons D. “Blocking αvβ3 Integrin Ligand Occupancy Inhibits the Progression of Albuminuria in Diabetic Rats,” J. Diabetes Res. 2014; 421827. Since amino acid 177-183 of β3 (Cysteine-loop) binding to heparin-binding domain (HBD) of vitronectin (VN) is considered necessary for an optimal response of vascular cells to IGF-I, see, Xi G, Maile L A, Yoo S E, Clemmons D R. “Expression of the human beta3 integrin subunit in mouse smooth muscle cells enhances IGF-I-stimulated signaling and proliferation,” J. Cell Physiol. 2008; 214(2): 306-315. Furthermore, Vascular Pharmaceuticals has reported that targeting the C-loop may inhibit IGF-1 signaling without triggering the potential negative effects of RGD-binding site antagonists (see WO2014036385).

In addition to modulation of cell adhesion, the integrin a αvβ3 is a receptor for the latency-associated peptides of transforming growth factors beta1 and beta3 and mediates TGF-beta activation, see, Ludbrook S B, Barry S T, Delves C J, Horgan C M. “The integrin alphavbeta3 is a receptor for the latency-associated peptides of transforming growth factors beta1 and beta3,” Biochem. J. 2003; Jan. 15, 369(Pt 2): 311-318. Recently, it was shown that integrin αvβ3 promotes myofibroblast differentiation by activating latent TGF-β1, see, Sarrazy V, Koehler A, Chow M L, Zimina E, Li C X, Kato H, Caldarone C A, Hinz B. “Integrins αvβ5 and αvβ3 promote latent TGF-β1 activation by human cardiac fibroblast contraction,” Cardiovasc. Res. 2014; Jun. 1, 102(3): 407-417.

### SUMMARY OF THE INVENTION

This invention relates to the treatment of chronic kidney disease, including diabetic nephropathy, focal segmental glomerulosclerosis (FSGS), nephrotic syndrome, non-diabetic chronic kidney disease, renal fibrosis and acute kidney injury by the administration of an RGD mimetic integrin receptor antagonist, either as a single agent or in combination with other agents.

The effects of Compound A (Example 1-18), a small molecule inhibitor, on urinary total protein/creatinine ratio, urinary albuminuria/creatinine ratio, renal histology, glomerular filtration rate, fibrosis score, gene expression, and function in a validated rodent diabetic nephropathy model ZSF-1 rats have been investigated. As described herein, the data demonstrates that Compound A (Example 1-18) showed renal protection by ameliorating proteinuria and albuminuria, improvement in markers of renal fibrosis and non-statistically significant improvements in glomerular filtration rate in ZSF-1 rats when compared to the untreated obese ZSF-1 rats. High doses of Compound A (Example 1-18) have also shown improvement of plasma TG and cholesterol in obese ZSF-1 rats compared to untreated obese ZSF-1 rats.

### DETAILED DESCRIPTION OF THE INVENTION

The instant invention relates to the treatment of chronic kidney disease, including diabetic nephropathy, focal segmental glomerulosclerosis (FSGS), nephrotic syndrome, non-diabetic chronic kidney disease, renal fibrosis, and acute kidney injury by the administration of an RGD mimetic integrin receptor antagonist, either as a single agent or in combination with other agents.

“RGD mimetic integrin receptor antagonist” as used herein refers to a non-selective integrin receptor antagonist that binds to the RGD site of integrins.

Nonlimiting examples of RGD mimetic integrin receptor antagonists include the following:

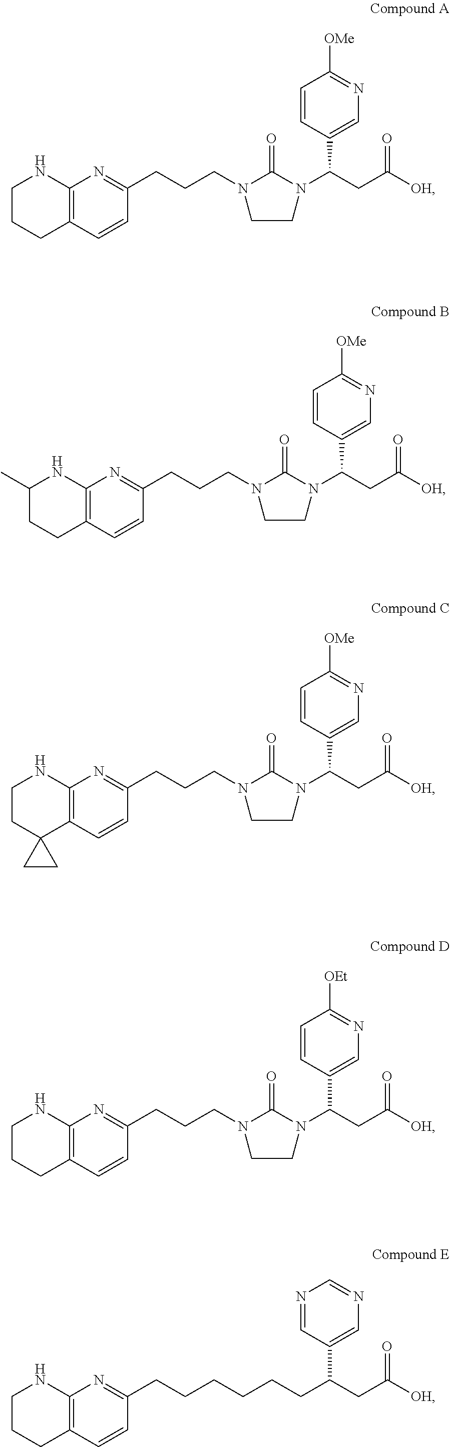 [SMILES: COc1ccc([C@H](CC(=O)O)N2CCN(CCCc3ccc4c(n3)NCCC4)C2=O)cn1 ; COc1ccc([C@H](CC(=O)O)N2CCN(CCCc3ccc4c(n3)NC(C)CC4)C2=O)cn1 ; COc1ccc([C@H](CC(=O)O)N2CCN(CCCc3ccc4c(n3)NCCC43CC3)C2=O)cn1 ; CCOc1ccc([C@H](CC(=O)O)N2CCN(CCCc3ccc4c(n3)NCCC4)C2=O)cn1 ; O=C(O)C[C@H](CCCCCCc1ccc2c(n1)NCCC2)c1cncnc1]

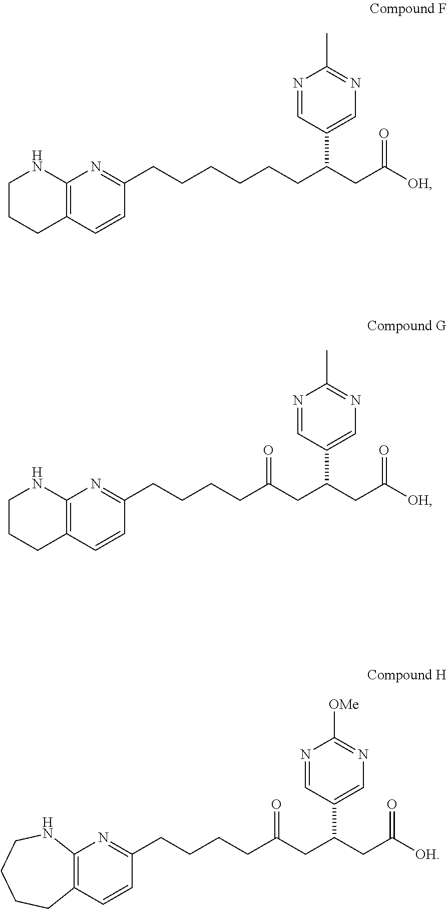 [SMILES: Cc1ncc([C@@H](CCCCCCc2ccc3c(n2)NCCC3)CC(=O)O)cn1 ; Cc1ncc([C@H](CC(=O)O)CC(=O)CCCCc2ccc3c(n2)NCCC3)cn1 ; COc1ncc([C@H](CC(=O)O)CC(=O)CCCCc2ccc3c(n2)NCCCC3)cn1]

Compound A is an RGD mimetic integrin receptor antagonist, and is also known as 3-{2-Oxo-3-[3-(5,6,7,8-tetrahydro-[1,8]naphthyridin-2-yl)-propyl]imidazolidin-1-yl}-3 (S)-(6-methoxy-pyridin-3-yl)-propionic acid, Example 1-18 or MK-0429. Compound A and its preparation are disclosed in U.S. Pat. Nos. 6,017,926; 6,262,268; 6,407,241; 6,423,845; 6,706,885; and 6,646,130; and in Nobuyoski Yasuda, et al. “An Efficient Synthesis of an αvß3 Antagonist,” J. Org. Chem. 2004, 69, 1959-1966, which are hereby incorporated by reference in their entirety. Hydroxylated metabolites of Compound A are disclosed in U.S. Pat. No. 6,426,353, which is hereby incorporated by reference in its entirety. Crystalline hydrates of Compound A are disclosed in U.S. Pat. No. 6,509,347, which is hereby incorporated by reference in its entirety.

Compound B is an RGD mimetic integrin receptor antagonist, which is disclosed in U.S. Pat. No. 6,472,403, which is hereby incorporated by reference in its entirety.

Compound C is an RGD mimetic integrin receptor antagonist, 3(S)-(6-Methoxy-pyridin-3-yl)-3-{2-oxo-3-(5,6,7,8-tetrahydro-5,5-ethyleno-[1,8]naphthyridin-2-yl)-propyl]-imidazolidin-1-yl}-propionic acid, which is disclosed in U.S. Pat. No. 6,472,403, which is hereby incorporated by reference in its entirety.

Compound D is an RGD mimetic integrin receptor antagonist, which is disclosed in U.S. Pat. No. 6,017,926, which is hereby incorporated by reference in its entirety.

Compound E is an RGD mimetic integrin receptor antagonist. Compound E and its preparation are disclosed in U.S. Pat. No. 6,297,249 and International Patent Publication WO 03/072042, which are hereby incorporated by reference in their entirety. Chiral intermediates of Compound E are disclosed in International Patent Publication WO 02/28840, which is hereby incorporated by reference in its entirety. TRIS salts of Compound E are disclosed in U.S. Pat. No. 6,750,220, which is hereby incorporated by reference in its entirety.

Compound F is an RGD mimetic integrin receptor antagonist, which is disclosed in U.S. Pat. No. 6,297,249, which is hereby incorporated by reference in its entirety.

Compound G is an RGD mimetic integrin receptor antagonist, which is disclosed in U.S. Pat. No. 6,410,526, which is hereby incorporated by reference in its entirety.

Compound H is an RGD mimetic integrin receptor antagonist. Compound H and its preparation are disclosed in U.S. Pat. No. 6,410,526 and International Patent Publication WO 02/028395, which are hereby incorporated by reference in their entirety.

“Diabetic nephropathy” is characterized by kidney damage or kidney disease caused by diabetes. Diabetic nephropathy is also known as Kimmelstiel-Wilson syndrome, or nodular diabetic glomerulosclerosis and intercapillary glomerulonephritis. It is a progressive kidney disease caused by angiopathy of capillaries in the kidney glomeruli, and is characterized by nephrotic syndrome and diffuse glomerulosclerosis. Diabetic nephropathy is often due to longstanding diabetes mellitus, and is a prime indication for dialysis in many developed countries. It is classified as a small blood vessel complication of diabetes.

“Focal segmental glomerulosclerosis (FSGS)” is a cause of nephrotic syndrome in children and adolescents, as well as an important cause of kidney failure in adults. It is also known as “focal glomerular sclerosis” or “focal nodular glomerulosclerosis” and accounts for about a sixth of the cases of nephrotic syndrome.

“Nephrotic syndrome” is a nonspecific kidney disorder characterized by a number of signs of disease: proteinuria, hypoalbuminemia and edema. It is characterized by an increase in permeability of the capillary walls of the glomerulus leading to the presence of high levels of protein passing from the blood into the urine; low levels of protein in the blood (hypoproteinemia or hypoalbuminemia), ascites and in some cases, edema; high cholesterol (hyperlipidaemia or hyperlipemia) and a predisposition for coagulation. The cause is damage to the glomeruli, which can be the cause of the syndrome or caused by it, that alters their capacity to filter the substances transported in the blood. The severity of the damage caused to the kidneys can vary and can lead to complications in other organs and systems. Kidneys affected by nephrotic syndrome have small pores in the podocytes, large enough to permit proteinuria (and subsequently hypoalbuminemia, <25 g/L, because some of the protein albumin has gone from the blood to the urine) but not large enough to allow cells through (hence no haematuria). “Non-diabetic chronic kidney disease,” also known as non-diabetic CKD and known as non-diabetic chronic renal disease, is a progressive loss in renal function over a period of months or years.

“Renal fibrosis” is the hallmark of chronic kidney disease, regardless of underlying etiology. The pathological finding of renal fibrosis is characterized by progressive tissue scarring including glomerulosclerosis, tubulointerstitial fibrosis and loss of renal parenchyma (including tubular atrophy, loss of capillaries and podocytes).

“Acute kidney injury,” also known as acute renal failure, is defined as an abrupt or rapid decline in renal filtration function. This condition is usually marked by a rise in serum creatinine concentration or by azotemia (a rise in blood urea nitrogen [BUN] concentration).

An embodiment of the invention includes a method for treating a disease selected from diabetic nephropathy, focal segmental glomerulosclerosis, nephrotic syndrome, non-diabetic kidney chronic kidney disease, renal fibrosis or acute kidney injury with an RGD mimetic integrin receptor antagonist. In a class of the embodiment, the disease is diabetic nephropathy. In another class of the embodiment, the disease is focal segmental glomerulosclerosis. In another class of the embodiment, the disease is nephrotic syndrome. In another class of the embodiment, the disease is non-diabetic kidney chronic kidney disease. In another class of the embodiment, the disease is renal fibrosis. In another class of the embodiment, the disease is acute kidney injury.

Another embodiment of the invention includes the use of RGD mimetic integrin receptor antagonist in the manufacture of a medicament for the treatment of a disease selected from diabetic nephropathy, focal segmental glomerulosclerosis, nephrotic syndrome, non-diabetic kidney chronic kidney disease, renal fibrosis or acute kidney injury with in a mammal in need thereof. In a class of the embodiment, the disease is diabetic nephropathy. In another class of the embodiment, the disease is focal segmental glomerulosclerosis. In another class of the embodiment, the disease is nephrotic syndrome. In another class of the embodiment, the disease is non-diabetic kidney chronic kidney disease. In another class of the embodiment, the disease is renal fibrosis. In another class of the embodiment, the disease is acute kidney injury.

### Dose and Routes of Administration

With regard to RGD mimetic integrin receptor antagonists of the invention, various preparation forms can be selected, and examples thereof include oral preparations such as tablets, capsules, powders, granules or liquids, or sterilized liquid parenteral preparations such as solutions or suspensions, suppositories, ointments and the like prepared with pharmaceutically acceptable carriers or diluents.

The term “pharmaceutically acceptable salt” as referred to in this description means ordinary, pharmaceutically acceptable salt. For example, when the compound has a hydroxyl group, or an acidic group such as a carboxyl group and a tetrazolyl group, then it may form a base-addition salt at the hydroxyl group or the acidic group; or when the compound has an amino group or a basic heterocyclic group, then it may form an acid-addition salt at the amino group or the basic heterocyclic group.

The base-addition salts include, for example, alkali metal salts such as sodium salts, potassium salts; alkaline earth metal salts such as calcium salts, magnesium salts; ammonium salts; and organic amine salts such as trimethylamine salts, triethylamine salts, dicyclohexylamine salts, ethanolamine salts, diethanolamine salts, triethanolamine salts, procaine salts, N,N′-dibenzylethylenediamine salts.

The acid-addition salts include, for example, inorganic acid salts such as hydrochlorides, sulfates, nitrates, phosphates, perchlorates; organic acid salts such as maleates, fumarates, tartrates, citrates, ascorbates, trifiuoroacetates; and sulfonates such as methanesulfonates, isethionates, benzenesulfonates, p-toluenesulfonates.

The term “pharmaceutically acceptable carrier or diluent” refers to excipients [e.g., fats, beeswax, semi-solid and liquid polyols, natural or hydrogenated oils, etc.]; water (e.g., distilled water, particularly distilled water for injection, etc.), physiological saline, alcohol (e.g., ethanol), glycerol, polyols, aqueous glucose solution, mannitol, plant oils, etc.); additives [e.g., extending agent, disintegrating agent, binder, lubricant, wetting agent, stabilizer, emulsifier, dispersant, preservative, sweetener, colorant, seasoning agent or aromatizer, concentrating agent, diluent, buffer substance, solvent or solubilizing agent, chemical for achieving storage effect, salt for modifying osmotic pressure, coating agent or antioxidant], and the like.

Solid preparations can be prepared in the forms of tablet, capsule, granule and powder without any additives, or prepared using appropriate carriers (additives). Examples of such carriers (additives) may include saccharides such as lactose or glucose; starch of corn, wheat or rice; fatty acids such as stearic acid; inorganic salts such as magnesium meta-silicate aluminate or anhydrous calcium phosphate; synthetic polymers such as polyvinylpyrrolidone or polyalkylene glycol; alcohols such as stearyl alcohol or benzyl alcohol; synthetic cellulose derivatives such as methylcellulose, carboxymethylcellulose, ethylcellulose or hydroxypropylmethylcellulose; and other conventionally used additives such as gelatin, talc, plant oil and gum arabic.

These solid preparations such as tablets, capsules, granules and powders may generally contain, for example, 0.1 to 100% by weight, and preferably 5 to 98% by weight, of the αvß3 RGD mimetic integrin receptor antagonist, based on the total weight of each preparation.

Liquid preparations are produced in the forms of suspension, syrup, injection and drip infusion (intravenous fluid) using appropriate additives that are conventionally used in liquid preparations, such as water, alcohol or a plant-derived oil such as soybean oil, peanut oil and sesame oil.

In particular, when the preparation is administered parenterally in a form of intramuscular injection, intravenous injection or subcutaneous injection, appropriate solvent or diluent may be exemplified by distilled water for injection, an aqueous solution of lidocaine hydrochloride (for intramuscular injection), physiological saline, aqueous glucose solution, ethanol, polyethylene glycol, propylene glycol, liquid for intravenous injection (e.g., an aqueous solution of citric acid, sodium citrate and the like) or an electrolytic solution (for intravenous drip infusion and intravenous injection), or a mixed solution thereof.

Such injection may be in a form of a preliminarily dissolved solution, or in a form of powder per se or powder associated with a suitable carrier (additive) which is dissolved at the time of use. The injection liquid may contain, for example, 0.1 to 10% by weight of an active ingredient based on the total weight of each preparation.

Liquid preparations such as suspension or syrup for oral administration may contain, for example, 0.1 to 10% by weight of an active ingredient based on the total weight of each preparation.

Each preparation in the invention can be prepared by a person having ordinary skill in the art according to conventional methods or common techniques. For example, a preparation can be carried out, if the preparation is an oral preparation, for example, by mixing an appropriate amount of the compound of the invention with an appropriate amount of lactose and filling this mixture into hard gelatin capsules which are suitable for oral administration. On the other hand, preparation can be carried out, if the preparation containing the compound of the invention is an injection, for example, by mixing an appropriate amount of the compound of the invention with an appropriate amount of 0.9% physiological saline and filling this mixture in vials for injection.

The components of this invention may be administered to mammals, including humans, either alone or, in combination with pharmaceutically acceptable carriers, excipients or diluents, in a pharmaceutical composition, according to standard pharmaceutical practice. The components can be administered orally or parenterally, including the intravenous, intramuscular, intraperitoneal, subcutaneous, rectal and topical routes of administration.

Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved. Further information about suitable dosages is provided below.

The term “administration” and variants thereof (e.g., “administering” a compound) in reference to a component of the invention means introducing the component or a prodrug of the component into the system of the animal in need of treatment. When a component of the invention or prodrug thereof is provided in combination with one or more other active agents, “administration” and its variants are each understood to include concurrent and sequential introduction of the component or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

A therapeutically effective amount of an RGD mimetic integrin receptor antagonist is administered to a patient undergoing treatment. In an embodiment, the RGD mimetic integrin receptor antagonist is administered in doses from about 25 mg to 1600 mg per day (including 25 mg, 50 mg, 100 mg, 200 mg, 400 mg, 800 mg, 1600 mg per day). In an embodiment of the invention, the RGD mimetic integrin receptor antagonist will be dosed QD or BID, with doses of 25 mg, 50 mg, 75 mg, 100 mg, 150 mg, 200 mg, 300 mg, 400 mg or 800 mg. In a class of the invention, the αvβ3 integrin antagonist will be dosed QD with doses of 25 mg, 50 mg, 75 mg, 100 mg, 150 mg, 200 mg, 300 mg, 400 mg or 800 mg. In another class of the invention, the RGD mimetic integrin receptor antagonist will be dosed BID with doses of 25 mg, 50 mg, 75 mg, 100 mg, 150 mg, 200 mg, 300 mg, 400 mg or 800 mg.

In a broad embodiment, any suitable additional active agent or agents, including but not limited to anti-hypertensive agents, anti-atherosclerotic agents, anti-diabetic agents and/or anti-obesity agents, may be used in any combination with an RGD mimetic integrin receptor antagonist in a single dosage formulation (a fixed dose drug combination), or may be administered to the patient in one or more separate dosage formulations which allows for concurrent or sequential administration of the active agents (co-administration of the separate active agents). Examples of the one or more additional active agents which may be employed include but are not limited to angiotensin converting enzyme inhibitors (e.g, alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril); dual inhibitors of angiotensin converting enzyme (ACE) and neutral endopeptidase (NEP) such as omapatrilat, sampatrilat and fasidotril; angiotensin II receptor antagonists, also known as angiotensin receptor blockers or ARBs, which may be in free-base, free-acid, salt or pro-drug form, such as azilsartan, e.g., azilsartan medoxomil potassium (EDARBI®), candesartan, e.g., candesartan cilexetil (ATACAND®), eprosartan, e.g., eprosartan mesylate (TEVETAN®), irbesartan (AVAPRO®), losartan, e.g., losartan potassium (COZAAR®), olmesartan, e.g, olmesartan medoximil (BENICAR®), telmisartan (MICARDIS®), valsartan (DIOVAN®), and any of these drugs used in combination with a thiazide-like diuretic such as hydrochlorothiazide (e.g., HYZAAR®, DIOVAN HCT®, ATACAND HCT®), etc.); potassium sparing diuretics such as amiloride HCl, spironolactone, epleranone, triamterene, each with or without HCTZ; carbonic anhydrase inhibitors, such as acetazolamide; neutral endopeptidase inhibitors (e.g., thiorphan and phosphoramidon); aldosterone antagonists; aldosterone synthase inhibitors; renin inhibitors (e.g. urea derivatives of di- and tri-peptides (See U.S. Pat. No. 5,116,835), amino acids and derivatives (U.S. Pat. Nos. 5,095,119 and 5,104,869), amino acid chains linked by non-peptidic bonds (U.S. Pat. No. 5,114,937), di- and tri-peptide derivatives (U.S. Pat. No. 5,106,835), peptidyl amino diols (U.S. Pat. Nos. 5,063,208 and 4,845,079) and peptidyl beta-aminoacyl aminodiol carbamates (U.S. Pat. No. 5,089,471); also, a variety of other peptide analogs as disclosed in the following U.S. Pat. Nos. 5,071,837; 5,064,965; 5,063,207; 5,036,054; 5,036,053; 5,034,512 and 4,894,437, and small molecule renin inhibitors (including diol sulfonamides and sulfinyls (U.S. Pat. No. 5,098,924), N-morpholino derivatives (U.S. Pat. No. 5,055,466), N-heterocyclic alcohols (U.S. Pat. No. 4,885,292) and pyrolimidazolones (U.S. Pat. No. 5,075,451); also, pepstatin derivatives (U.S. Pat. No. 4,980,283) and fluoro- and chloro-derivatives of statone-containing peptides (U.S. Pat. No. 5,066,643); enalkrein; RO 42-5892; A 65317; CP 80794; ES 1005; ES 8891; SQ 34017; aliskiren (2(S),4(S),5(S),7(S)—N-(2-carbamoyl-2-methylpropyl)-5-amino-4-hydroxy-2,7-diisopropyl-8-[4-methoxy-3-(3-methoxypropoxy)-phenyl]-octanamid hemifumarate) SPP600, SPP630 and SPP635); endothelin receptor antagonists; vasodilators (e.g. nitroprusside); calcium channel blockers (e.g., amlodipine, nifedipine, verapamil, diltiazem, felodipine, gallopamil, niludipine, nimodipine, nicardipine, bepridil, nisoldipine); potassium channel activators (e.g., nicorandil, pinacidil, cromakalim, minoxidil, aprilkalim, loprazolam); sympatholitics; beta-adrenergic blocking drugs (e.g., acebutolol, atenolol, betaxolol, bisoprolol, carvedilol, metoprolol, metoprolol tartate, nadolol, propranolol, sotalol, timolol); alpha adrenergic blocking drugs (e.g., doxazocin, prazocin or alpha methyldopa); central alpha adrenergic agonists; peripheral vasodilators (e.g. hydralazine); nitrates or nitric oxide donating compounds, e.g. isosorbide mononitrate; lipid lowering agents, e.g., HMG-CoA reductase inhibitors such as simvastatin and lovastatin which are marketed as ZOCOR® and MEVACOR® in lactone pro-drug form and function as inhibitors after administration, and pharmaceutically acceptable salts of dihydroxy open ring acid HMG-CoA reductase inhibitors such as atorvastatin (particularly the calcium salt sold in LIPITOR®), rosuvastatin (particularly the calcium salt sold in CRESTOR®), pravastatin (particularly the sodium salt sold in PRAVACHOL®), and fluvastatin (particularly the sodium salt sold in LESCOL®); a cholesterol absorption inhibitor such as ezetimibe (ZETIA®), and ezetimibe in combination with any other lipid lowering agents such as the HMG-CoA reductase inhibitors noted above and particularly with simvastatin (VYTORIN®) or with atorvastatin calcium; niacin in immediate-release or controlled release forms, and particularly niacin in combination with a DP antagonist such as laropiprant and/or with an HMG-CoA reductase inhibitor; niacin receptor agonists such as acipimox and acifran, as well as niacin receptor partial agonists; metabolic altering agents including insulin sensitizing agents and related compounds for the treatment of diabetes such as biguanides (e.g., metformin), meglitinides (e.g., repaglinide, nateglinide), sulfonylureas (e.g., chlorpropamide, glimepiride, glipizide, glyburide, tolazamide, tolbutamide), thiazolidinediones also referred to as glitazones (e.g., pioglitazone, rosiglitazone), alpha glucosidase inhibitors (e.g., acarbose, miglitol), dipeptidyl peptidase inhibitors, (e.g., sitagliptin (JANUVIA®), alogliptin, vildagliptin, saxagliptin, linagliptin, dutogliptin, gemigliptin), ergot alkaloids (e.g., bromocriptine), combination medications such as JANUMET® (sitagliptin with metformin), and injectable diabetes medications such as exenatide and pramlintide acetate; phosphodiesterase-5 (PDE5) inhibitors such as sildenafil (Revatio, Viagra), tadalafil (Cialis, Adcirca) vardenafil HCl (Levitra); inhibitors of glucose uptake, such as sodium-glucose transporter (SGLT) inhibitors and its various isoforms, such as SGLT-1, SGLT-2 (e.g., ASP-1941, TS-071, BI-10773, tofogliflozin, LX-4211, canagliflozin, dapagliflozin, ertugliflozin, ipragliflozin and remogliflozin), and SGLT-3; a stimulator of soluble guanylate cyclase (sGC), such as riociguat, vericiguat; or with other drugs beneficial for the prevention or the treatment of the above-mentioned diseases including but not limited to diazoxide; and including the free-acid, free-base, and pharmaceutically acceptable salt forms, pro-drug forms (including but not limited to esters), and salts of pro-drugs of the above medicinal agents where chemically possible. Trademark names of pharmaceutical drugs noted above are provided for exemplification of the marketed form of the active agent(s); such pharmaceutical drugs could be used in a separate dosage form for concurrent or sequential administration with a compound of the instant invention, or the active agent(s) therein could be used in a fixed dose drug combination including a compound of the instant invention.

An embodiment of the invention includes a method for treating a disease selected from diabetic nephropathy, focal segmental glomerulosclerosis, nephrotic syndrome, renal fibrosis, acute kidney injury or non-diabetic kidney chronic kidney disease with an RGD mimetic integrin receptor antagonist and an additional agent selected from an anti-hypertensive agent, anti-atherosclerotic agent, anti-diabetic agent and/or anti-obesity agent. In a class of the embodiment, the additional agent is selected from an angiotensin converting enzyme inhibitors; dual inhibitor of angiotensin converting enzyme (ACE) and neutral endopeptidase (NEP); angiotensin II receptor antagonist; a thiazide-like diuretic; potassium sparing diuretic; carbonic anhydrase inhibitor; neutral endopeptidase inhibitor; aldosterone antagonist; aldosterone synthase inhibitor; renin inhibitor; endothelin receptor antagonist; vasodilator; calcium channel blocker; potassium channel activator; sympatholitics; beta-adrenergic blocking drug; alpha adrenergic blocking drug; nitrate; nitric oxide donating compound; lipid lowering agent; a cholesterol absorption inhibitor; niacin; niacin receptor agonist; niacin receptor partial agonist; metabolic altering agent; alpha glucosidase inhibitor; dipeptidyl peptidase inhibitor; ergot alkaloids; phosphodiesterase-5 (PDE5) inhibitor; or a combination thereof. In a subclass of the embodiment, the additional agent is enalapril. In another subclass of the embodiment, the additional agent is losartan. In another subclass of the embodiment, the additional agents are enalapril and losartan.

Both angiotensin-converting enzyme inhibitors (ACEi) and angiotensin receptor blockers (ARBs) are standard of care for the treatment of patients with diabetic nephropathy. In general, an ACE inhibitor, for example enalapril, is dosed 2.5 mg to 20 mg/BID in doses consisting of, but not limited to, 2.5 mg, 5 mg, 7.5 mg, 10 mg, 12.5 mg, 15 mg, 17.5 mg, 20 mg BID (twice daily). In general, an ARBs, for example losartan, is dosed 25 mg to 100 mg/day in doses consisting of, but not limited to, 25 mg, 50 mg, 75 mg, 100 mg QD (once daily) for reduction of proteinuria and control of blood pressure. In another embodiment of the intervention, the combination therapy of RGD mimetic integrin receptor antagonist Compound A (doses from 25 mg to 800 mg BID) in doses consisting of, but not limited to, 25 mg, 50 mg, 75 mg, 100 mg, 150 mg, 200 mg, 300 mg, 400 mg, 800 mg BID with an ACE inhibitor, such as enalapril (doses from 2.5 mg to 20 mg BID) in doses consisting of but not limited to 2.5 mg, 5 mg, 7.5 mg, 10 mg, 12.5 mg, 15 mg, 17.5 mg, 20 mg BID or with an ARB, such as losartan (doses from 25 mg to 100 mg/day) in doses consisting but not limited to 25 mg, 50 mg, 75 mg, 100 mg QD are administered to patients with diabetic nephropathy.

Enalapril is an ACE inhibitor used to treat high blood pressure (hypertension) in adults and children who are at least 1 month old, and congestive heart failure in adults. It is also used for treatment of chronic kidney disease. Enalapril maleate is the maleate salt of enalapril, and is supplied as 2.5 mg, 5 mg, 10 mg and 20 mg tablets for oral administration.

Losartan is an angiotensin II receptor antagonist used to keep blood vessels from narrowing, which lowers blood pressure and improves blood flow. Losartan potassium is the potassium salt of losartan and is used to treat high blood pressure (hypertension). It is also used to lower the risk of stroke in certain people with heart disease and slow long-term kidney damage in people with type 2 diabetes who also have high blood pressure. Losartan potassium is supplied as 25 mg, 50 mg and 100 mg tablets for oral administration.

In the Schemes and Examples below, various reagent symbols and abbreviations have the following meanings:
AcOH: Acetic acid
9-BBN: 9-Borabicyclo[3.3.1]nonane
BINAL-H: 1,1-bi-2,2′-naphthol-lithium aluminum hydride complex
BINOL: 1,1′-Bi-2-naphthol
BOC(Boc): t-Butyloxycarbonyl
BSA: Bovine Serum Albumin
CBZ(Cbz): Carbobenzyloxy or benzyloxycarbonyl
DBU: 1,8-Diazabicyclo[5.4.0]undec-7-ene
DCM: Dichloromethane
DEAD: Diethyl azodicarboxylate
DIBAH or
DIBAL-H: Diisobutylaluminum hydride
DIPEA: Diisopropylethylamine
DMAP: 4-Dimethylaminopyridine
DME: 1,2-Dimethoxyethane
DMF: N,N-Dimethylformamide
DMSO: Dimethylsulfoxide
DPPF: 1,1′-Bis(diphenylphosphino)-ferrocene
Et₃N: Triethylamine
EtOAc: Ethyl acetate
EtOH: Ethanol
HMPA: Hexamethylphosphoramide
HOAc: Acetic acid
HPLC: High-performance liquid chromatography
iPAc Isopropyl acetate
LAH: Lithium aluminum hydride
LDA: Lithium diisopropylamide
m-CPBA: meta-Chloroperoxybenzoic acid
MeOH: Methanol
MNNG: 1,1-methyl-3-nitro-1-nitrosoguanidine
MTBE: Methyl tert-butyl ether
NaBH₃CN: Sodium cyanoborohydride
NaOAc: Sodium acetate
NMP: N-Methyl-2-pyrrolidone
Pd/C: Palladium on activated carbon catalyst
Ph: Phenyl
PPh₃: Triphenylphosphine
TBS: Tris-buffered saline
TBTU: O-Benzotriazol-1-yl-N,N,N′,N′-tetramethyluronium tetrafluoroborate.
p-TSA: p-Toluenesulfonic acid
p-TsOH: p-Toluenesulfonic acid
TFA: Trifluoroacetic acid
THF: Tetrahydrofuran
TLC: Thin Layer Chromatography
TMEDA: N,N,N′,N′-Tetramethylethylenediamine
TMS: Trimethylsilyl

“Solka Floc®” is the brand name powdered cellulose that is carefully processed, highly purified functional cellulose. “Celite®”, also known as celite, is diatomaceous earth.

The compounds of the present invention can be prepared according to the procedures of the following reaction Schemes and Examples, or modifications thereof, using readily available starting materials, reagents, and, where appropriate, conventional synthetic procedures. In these procedures, it is also possible to make use of variants which are themselves known to those of ordinary skill in the organic synthetic arts, but are not mentioned in greater detail.

### Example 1

### Synthesis of Compound A (1-18)

### Step A: Preparation of Compound 1-4

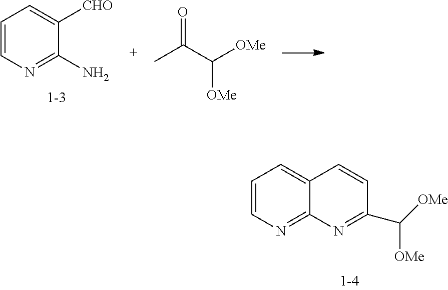

To a cold (6° C.) solution of 2-amino-3-formylpyridine 1-3 (40 g, 0.316 mol), ethanol (267 ml), water (41 ml), and pyruvic aldehyde dimethyl acetal (51.3 ml, 0.411 mol) was added 5 M NaOH (82.3 ml, 0.411 mol) at a rate such that the internal temperature was lower than 20° C. After stirring at ambient temperature for 1 hour, the ethanol was removed under vacuum, and iPAc (100 mL) and NaCl (55 g) were added. The layers were separated and the aqueous layer was extracted with iPAc (2×100 ml). The organic layers were combined, filtered through a silica gel bed (90 g), followed by rinse with iPAc (1 L). The fractions were combined and concentrated to 200 ml at 38° C. To the solution was slowly added hexane (400 ml). The resulting suspension was cooled to 10° C. and aged for 30 min before filtration. The suspension was filtered and dried under vacuum to give the product 1-4 (54.2 g; 84%) as colorless crystals; m.p. 53.5-55.5° C. To the mother liquors was added additional hexane (100 mL), and another 7.2 g (11%) of 1-4 was isolated after filtration.

¹H NMR (300 MHz; CDCl₃): δ 8.89 (dd, J=4.3 and 2.0 Hz, 1H), 8.03 (d, J=8.4 Hz, 1H), 7.98 (dd, J=8.1 and 2.0 Hz, 1H), 7.56 (d, J=8.4 Hz, 1H), 7.26 (dd, J=8.1 and 4.3 Hz, 1H), 5.28 (s, 1H), and 3.30 (s, 6H).

¹³C NMR (75.5 MHz; CDCl₃): δ 161.3, 155.0, 153.5, 137.9, 136.8, 122.5, 122.3, 119.4, 105.9, and 54.9.

### Step B: Preparation of Compound 1-5

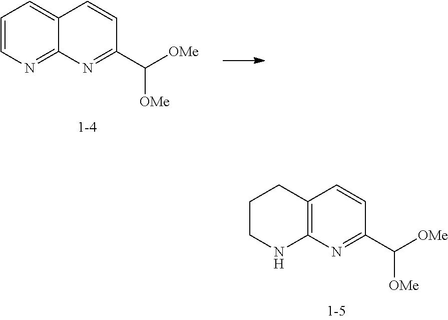 [SMILES: COC(OC)c1ccc2cccnc2n1 ; COC(OC)c1ccc2c(n1)NCCC2]

A solution of the acetal 1-4 (20.0 g; 97.9 mmol) in ethanol (400 mL) was hydrogenated in the presence of PtO₂ (778 mg) under one atmospheric pressure of hydrogen at room temperature for 18 hours. The reaction mixture was filtered through Solka Floc® and washed with a mixture of ethanol-H₂O (1:2 v/v). The filtrate and washings were combined and concentrated in vacuo to remove ethanol. The product crystallized as the ethanol was removed. The crystals were filtered and dried in vacuo to give product 1-5 (18.7 g, 92%); m.p. 91-92.5° C. ¹H NMR (300 MHz; CDCl₃): δ 7.08 (d, J=7.4 Hz, 1H), 6.62 (d, J=7.4 Hz, 1H), 5.07 (s, 2H; 1H exchangeable with D₂O), 3.37-3.29 (m, 2H), 3.29 (s, 6H), 2.64 (t, J=6.3 Hz, 2H), and 1.86-1.78 (m, 2H).

¹³C NMR (75.5 MHz; CDCl₃): δ 155.9, 153.0, 136.3, 116.0, 109.8, 103.9, 53.3, 41.5, 26.6, and 21.2.

### Step C: Preparation of Compound 1-6

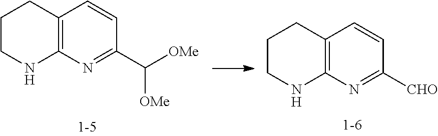 [SMILES: COC(OC)c1ccc2c(n1)NCCC2 ; O=Cc1ccc2c(n1)NCCC2]

To a mixture of the acetal 1-5 (35 g, 0.16 mol) in cold water (˜5° C., 90 ml) was added concentrated aqueous HCl (30 ml, 0.36 mol). The resulting solution was heated at 85° C. for 2.5 h. After the reaction was cooled to 13° C., iPAc (60 ml) was added. To the mixture was added aqueous NaOH (50 wt %) slowly to about pH 11, keeping the internal temperature below 25° C. The layers were separated and the aqueous layer was extracted with iPAc (2×120 ml). The organic layers were combined and concentrated in vacuo to give a reddish oil (26 g; 87.5 wt %; 95.3%) which was used in next reaction without further purification. An authentic sample was prepared by crystallization from THF; m.p. 63.5-64° C.

¹H NMR (300 MHz; CDCl₃): δ 9.70 (s, 1H), 7.17 (d, J=7.3 Hz, 1H), 7.03 (d, J=7.3 Hz, 1H), 5.94 (bs, 1H), 3.39-3.33 (m, 2H), 2.69 (t, J=6.3 Hz, 2H), and 1.84-1.80 (m, 2H).

¹³C NMR (75.5 MHz; CDCl₃): δ 192.8, 156.8, 149.5, 136.2, 122.5, 113.4, 41.4, 27.2, and 20.6.

### Step D: Preparation of Compound 1-7

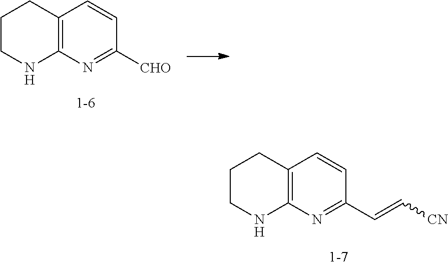 [SMILES: O=Cc1ccc2c(n1)NCCC2 ; N#CC=Cc1ccc2c(n1)NCCC2]

To a solution of the aldehyde 1-6 (26.0 g, 87.5 wt %; 140 mmol) and diethyl (cyanomethyl)phosphonate (26.7 mL; 140 mmol) in THF (260 ml) was added 50 wt % aqueous NaOH (14.8 g; 174 mmol) at a rate such that the internal temperature was below 26° C. After stirring at room temperature 1 hour, 260 ml of iPAc was added. The organic layer was separated and concentrated in vacuo to give 1-7 as a yellow solid (31.6 g, 84.6 wt %, 90% yield from 1-5, trans:cis ˜9:1). Authentic samples (trans and cis) were purified by silica gel column chromatography.

trans-1-7: m.p. 103.7-104.2° C.;

¹H NMR (300 MHz; CDCl₃): δ 7.14 (d, J=16.0 Hz, 1H), 7.12 (d, J=7.2 Hz, 1H), 6.48 (d, J=7.2 Hz, 1H), 6.33 (d, J=16.0 Hz, 1H), 5.12 (bs, 1H), 3.41-3.36 (m, 2H), 2.72 (t, J=6.3 Hz, 2H), and 1.93-1.84 (m, 2H).

¹³C NMR (75.5 MHz; CDCl₃): δ 156.1, 149.4, 147.4, 136.3, 120.1, 118.8, 114.8, 97.7, 41.4, 27.0, and 21.0.

cis-1-7:

¹H NMR (300 MHz; CDCl₃): δ 7.09 (d, J=7.3 Hz, 1H), 6.87 (d, J=11.8 Hz, 1H), 6.73 (d, J=7.3 Hz, 1H), 5.35 (d, J=11.8 Hz, 1H), 3.37-3.33 (m, 2H), 2.69 (t, J=6.3 Hz, 2H), and 1.90-1.81 (m, 2H).

¹³C NMR (75.5 MHz; CDCl₃): δ 155.5, 147.8, 147.4, 136.0, 119.1, 117.3, 114.2, 95.8, 41.2, 26.7, and 20.8.

### Step E: Preparation of Compound 1-8

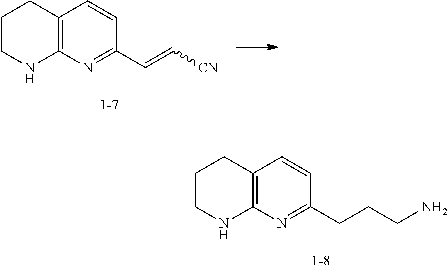 [SMILES: N#CC=Cc1ccc2c(n1)NCCC2 ; NCCCc1ccc2c(n1)NCCC2]

A slurry of the nitrile 1-7 (648 g; 3.5 mol) and saturated aqueous ammonium hydroxide (7 L) was hydrogenated under 40 psi of hydrogen at 50° C. for 16 h in the presence of Raney nickel 2800 (972 g). The mixture was filtered through Solka Floc® and the pad was rinsed with water (2×1 L). After addition of NaCl (3.2 kg), the mixture was extracted with CH₂Cl₂ (3×5 L). The combined organic phases were concentrated to an oil. The oil was dissolved in MTBE (1 L) and seeded. The suspension was slowly evaporated to provide the amine 1-8 as a colorless crystalline solid (577 g; 89%); m.p. 66.0-68.5° C.

¹H NMR (400 MHz; CDCl₃): δ 7.03 (d, J=7.3 Hz, 1H), 6.33 (d, J=7.3 Hz, 1H), 4.88 (bs, 1H), 3.37 (t, J=5.3 Hz, 2H), 2.72 (t, J=6.9 Hz, 2H), 2.67 (t, J=6.3 Hz, 2H), 2.57 (t, J=7.5 Hz, 2H), 1.92-1.74 (m, 6H).

¹³C NMR (101 MHz; CDCl₃): δ 157.9, 155.7, 136.6, 113.1, 111.2, 41.8, 41.5, 35.1, 33.7, 26.3, and 21.5.

### Step F: Preparation of Compound 1-10

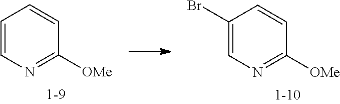 [SMILES: COc1ccccn1 ; COc1ccc(Br)cn1]

To a suspension of 2-methoxypyridine (1-9) (3.96 kg; 36.3 mol), NaOAc (3.57 kg; 39.9 mol), and dichloromethane (22 L) was added a solution of bromine (2.06 L; 39.9 mol) in dichloromethane (2 L), maintaining the reaction temperature below 7° C. over 2-3 hours. The mixture was aged for 1 hour at 0° C.-7° C. and stirred at room temperature overnight. The reaction mixture was filtered and rinsed with dichloromethane (about 5 L) (the filtration step may be omitted without negatively impacting the yield). The filtrate and washings were combined, washed with cold 2 M NaOH (22 L; pH is maintained between 9 and 10) maintaining the temperature below 10° C., and with cold water (11 L). The organic layer was separated and concentrated under reduced pressure to give crude product 1-10 (6.65 kg). The crude product 1-10 was purified by vacuum distillation to give pure 1-10 (5.90 kg, 86%). (Reference: G. Butora et al., J. Amer. Chem. Soc. 1997, 119, 7694-7701).

¹H NMR (250 MHz; CDCl₃): δ 8.18 (d, J=2.5 Hz, 1H), 7.61 (dd, J=8.8 and 2.5 Hz, 1H), 6.64 (d, J=8.8 Hz, 1H), and 3.89 (s, 3H).

¹³C NMR (62.9 MHz; CDCl₃): δ 162.9, 147.5, 141.0, 112.6, 111.7, and 53.7.

### Step G: Preparation of Compound 1-11

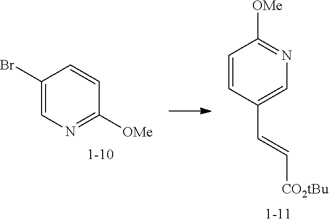 [SMILES: COc1ccc(Br)cn1 ; COc1ccc(/C=C/C(=O)OC(C)(C)C)cn1]

A mixture of tert-butyl acrylate (98%; 137 mL; 916 mmol), triethylamine (100 mL; 720 mmol), tri-O-tolylphosphine (97%; 6.30 g; 20 mmol), Pd(OAc)₂ (1.80 g; 8 mmol), and NMP (90 mL) was degassed three times. The mixture was heated to 90° C. and a solution of 2-methoxy-5-bromopyridine 1-10 (50.0 g; 266 mmol) and NMP (10 mL) was added via addition funnel over 1 hour, maintaining the reaction temperature at 90° C. The reaction was heated for 12 hours after complete addition. The reaction mixture was cooled down to room temperature after completion of the reaction. To the reaction mixture was added toluene (400 mL) and the resulting solution was then passed through a pad of Solka Floc®. The filter cake was washed with toluene (270 mL). The toluene solution was washed three times with water (540 mL, each). An aqueous solution of NaOCl (2.5%; 200 mL) was slowly added to the toluene solution keeping the temperature about 30° C. The reaction was aged 50 min with vigorous stirring. The organic layer was separated, washed with water (540 mL) three times, and followed by saturated aqueous NaCl (270 mL). The organic layer was concentrated to an oil. The oil was dissolved in 270 mL hexanes and loaded onto a silica gel (90 g) pad. The silica gel pad was washed with hexanes (73 mL). The product 1-11 was eluted with EtOAc:hexane (1:8; v/v) in about 730 mL. The yellow solution was concentrated to an oil (126 g; 49.2 wt %; 98.4% yield). The crude oil was used for the next reaction without further purification. Authentic crystalline material was obtained by further concentration of the oil; m.p. 44-45° C.

¹H NMR (250 MHz; CDCl₃): δ 8.23 (d, J=2.4 Hz, 1H), 7.73 (dd, J=8.7 and 2.4 Hz, 1H), 7.50 (d, J=16.0 Hz, 1H), 6.73 (d, J=8.7 Hz, 1H), 6.25 (d, J=16.0 Hz, 1H), 3.94 (s, 3H), and 1.51 (s, 9H).

¹³C NMR (62.9 MHz; CDCl₃): δ 166.1, 165.1, 148.1, 139.9, 136.3, 124.0, 119.1, 111.5, 80.6, 53.7, and 28.2.

### Step H: Preparation of Compound 1-12

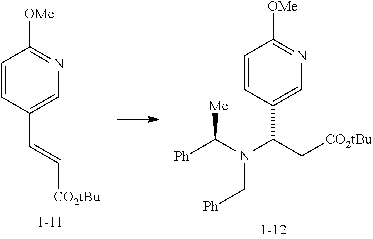

To a solution of (R)-(+)-N-benzyl-α-methylbenzylamine (88 mL; 0.42 mol) and anhydrous THF (1 L) was added n-BuLi (2.5 M in hexanes; 162 mL; 0.41 mol) over 1 hour at −30° C. The solution was then cooled to −65° C. A solution of t-butyl ester 1-11 (65.9 g; 0.28 mol) in anhydrous THF (0.5 L) was added over 90 minutes during which the temperature rose to −57° C. After the reaction was complete, the reaction solution was poured into a mixture of saturated aqueous NH₄Cl (110 mL) and EtOAc (110 mL). The organic layer was separated, washed separately with aqueous AcOH (10%; 110 mL), water (110 mL) and saturated aqueous NaCl (55 mL). The organic layer was concentrated in vacuo to a crude oil. The crude oil was purified by passing through a silica gel (280 g) pad eluting with a mixture of EtOAc and hexanes (5:95). The fractions containing the product were combined and concentrated in vacuo to give a thick oil. The resulting oil was used directly in the next step. The oil contained 91 g (0.20 mol, 73% yield) of the product 1-12.

¹H NMR (400 MHz; CDCl₃): δ 8.16 (d, J=2.4 Hz, 1H), 7.65 (dd, J=8.8 and 2.4 Hz, 1H), 7.40 (m, 2H), 7.34 (m, 2H), 7.30-7.16 (m, 6H), 6.74 (d, J=8.8 Hz, 1H), 4.39 (dd, J=9.8 and 5.3 Hz, 1H), 3.97 (q, J=6.6 Hz, 1H), 3.94 (s, 3H), 3.67 (s, 2H), 2.52 (dd, J=14.9 and 5.3 Hz, 1H), 2.46 (dd, J=14.9 and 9.8 Hz, 1H), 1.30 (d, J=6.6 Hz, 3H), and 1.26 (s, 9H);

¹³C NMR (101 MHz; CDCl₃): δ 170.8, 163.3, 146.4, 143.8, 141.3, 138.6, 130.0, 128.24, 128.19, 127.9, 127.7, 127.0, 126.6, 110.4, 80.5, 57.4, 56.6, 53.4, 50.7, 37.5, 27.8, and 17.3.

### Step I: Preparation of Compound 1-13

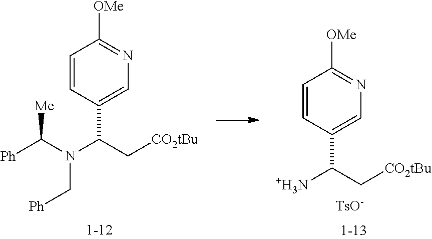

The thick oil (1-12; containing 80.3 g; 0.18 mol) was hydrogenated in the presence of Pd(OH)₂ (20 wt % on carbon; 8.0 g) in a mixture of EtOH (400 mL), AcOH (40 mL), water (2 mL) under 40 psi of hydrogen at 35° C. for 8 hours. The reaction mixture was filtered through a pad of Solka Floc®, evaporated to a thick oil in vacuo, and flushed with MTBE (2 L each) several times. Upon cooling, the batch solidified to a thick white solid. The thick slurry was heated to 50° C. and the solids dissolved. A hot solution (40° C.) of p-TsOH (41.7 g; 0.22 mol) and MTBE (400 mL) was then transferred slowly to the warm solution of the amine. After about 30% of the p-TsOH solution had been added, the solution was seeded and a thick slurry formed. The addition was continued and was complete in 2 hours. The solution was aged after completion of the addition for 3 hours at 45° C. The solution was then slowly cooled to room temperature. The solution was aged for 12 hours at room temperature and then cooled to 6° C. The very thick slurry was filtered, washed with MTBE (100 mL) and dried under vacuum at 35° C. for several days to give the product 1-13 (71.0 g; 73%); mp: 142-144° C.

¹H NMR (400 MHz; CDCl₃): δ 8.40 (bs, 3H), 8.22 (s, 1H), 7.87 (d, J=8.8 Hz, 1H), 7.56 (d, J=8.0 Hz, 2H), 7.11 (d, J=8.0 Hz, 2H), 6.65 (d, J=8.8 Hz, 1H), 4.63 (m, 1H), 3.91 (s, 3H), 3.09 (dd, J=16.5 and 6.0 Hz, 1H), 2.87 (dd, J=16.5 and 8.8 Hz, 1H), 2.36 (s, 3H), and 1.27 (s, 9H);

¹³C NMR (101 MHz; CDCl₃): δ 168.4, 164.2, 146.8, 140.9, 140.4, 137.8, 128.8, 125.8, 124.3, 111.0, 81.6, 53.5, 49.6, 39.3, 27.8, and 21.3.

### Step J: Preparation of Compound 1-14

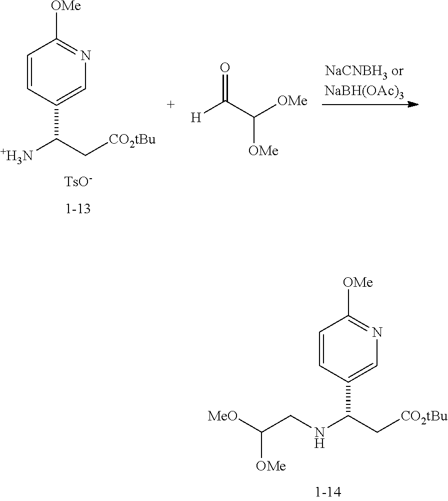

Method A: Reductive Amination with Sodium Cyanoborohydride

To a mixture of p-TSA salt 1-13 (50 g; 0.118 mol), MeOH (300 mL), and glyoxal-1,1-dimethyl acetal (45 wt % in MTBE; 40 g; 0.165 mol) was slowly added a solution of NaBH₃CN (9.35 g; 0.141 mol; 95%) in MeOH (50 mL). The rate of addition was such that the temperature never exceeded 3.5° C. (over 50 min). The reaction mixture was allowed to warm up to ambient temperature. After reaction completion (4-5 hours, final batch temperature was 16° C.), ice was placed around the flask and aqueous NaHCO₃ (14.8 g in 200 mL of H₂O) solution was added slowly. The mixture was concentrated to 420 mL. Additional H₂O (200 mL) and EtOAc (500 mL) were added. The aqueous layer was separated and extracted with EtOAc (500 mL). The organic layers were combined, dried over MgSO₄, and concentrated to approximately 100 mL. The resulting solution was passed through a small silica gel pad followed by additional 300 mL of EtOAc. The fractions containing 1-14 were combined and concentrated in vacuo to give 46.2 g of product 1-14 (46.2 g; 90.4 wt %; 92%) as an oil. This compound was used for the next step without further purification. An authentic sample was prepared by silica gel column chromatography.

¹H NMR (400 MHz; CDCl₃): δ 8.08 (d, J=2.4 Hz, 1H), 7.61 (dd, J=8.4 and 2.4 Hz, 1H), 6.73 (d, J=8.4 Hz, 1H), 4.41 (t, J=5.6 Hz, 1H), 4.00 (dd, J=8.2 and 6.0 Hz, 1H), 3.93 (s, 3H), 3.35 (s, 3H), 3.31 (s, 3H), 2.67 (dd, J=15.3 and 8.2 Hz, 1H), 2.60 (dd, J=12.0 and 5.6 Hz, 1H), 2.51 (dd, J=12.0 and 5.6 Hz, 1H), 2.49 (dd, J=15.3 and 6.0 Hz, 1H), and 1.40 (s, 9H);

¹³C NMR (101 MHz, CDCl₃): δ 170.6, 163.8, 145.9, 137.4, 130.4, 110.9, 103.5, 80.9, 56.9, 53.71, 53.68, 53.4, 48.6, 43.8, and 28.0.

Method B: Reductive Amination with Sodium Triacetoxyborohydride

To a solution of p-TSA salt 1-13 (100 g; 0.239 mmol) and glyoxal-1,1-dimethyl acetal (60 wt % in water; 39.3 mL; 0.261 mol) in THF (400 mL) was slowly added a suspension of sodium triacetoxyborohydride (79 g; 0.354 mol) in THF (200 mL) maintaining the batch temperature below 10° C. After the addition was complete, the suspension was rinsed with THF (40 mL) and added to the reaction mixture. The mixture was aged at 5-10° C. for 30 minutes and then at ambient temperature for 30 minutes. The mixture was cooled down to below 10° C. To the mixture was added aqueous sodium carbonate solution (1.2 L, 10 wt %), maintaining the batch temperature below 10° C. To the mixture was added EtOAc (750 mL). The organic layer was separated, washed with saturated aqueous sodium hydrogencarbonate (600 mL) and then water (500 mL). The organic layer was concentrated in vacuo and flushed with EtOAc to remove remaining water. The mixture was flushed with THF to remove residual EtOAc and the THF solution was used for the next reaction. The solution contained 74.1 g (92.2% yield) of the product 1-14.

### Step K: Preparation of Compounds 1-15 and 1-16

### Method A:

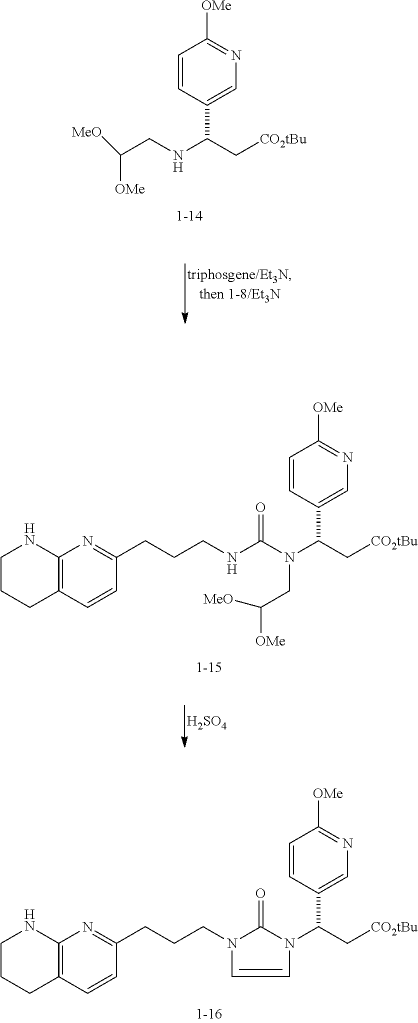

To a cold (−10° C.) solution of bis(trichloromethyl)carbonate (triphosgene) (3.0 g; 9.8 mmol) in anhydrous THF (60 mL) was slowly added a solution of acetal 1-14 (9.5 g; 85 wt %; 24 mmol) and triethylamine (4.4 mL; 32 mmol) in anhydrous THF (35 mL), keeping the reaction temperature below 5° C. The reaction mixture was aged at 5° C. for 30 minutes and at ambient temperature for 30 minutes. The excess phosgene was purged from the reaction mixture with a helium sparge through a scrubber containing aqueous NaOH. To the mixture was added anhydrous THF (20 mL). To the resulting suspension was added amine 1-8 (5.3 g; 94 wt %; 26 mmol) and triethylamine (4.4 mL; 32 mmol) at 5° C. The suspension was stirred at 40° C. for 6 hours. The reaction mixture was cooled to ambient temperature and 2 M aqueous sulfuric acid (30 mL) was added to the mixture at 22° C. The mixture was stirred at ambient temperature for 10 hours. The reaction mixture was added to a mixture of iPAc (50 mL) and 2 M aqueous sulfuric acid (15 mL). The aqueous layer was separated and washed with iPAc (50 mL). To the aqueous layer was added iPAc (50 mL) and the pH of the aqueous layer was adjusted to 8.2 by addition of solid Na₂CO₃. The organic layer was separated, washed with dilute aqueous NaCl (33 mL) twice, and concentrated in vacuo to give crude 1-16 as an oil (24.7 g; 40.1 wt %; 85%). An authentic sample was purified by silica gel column chromatography as an oil.

¹H NMR (400 MHz; CDCl₃): δ 8.13 (d, J=2.8 Hz, 1H), 7.60 (dd, J=8.8 and 2.8 Hz, 1H), 7.04 (d, J=7.2 Hz, 1H), 6.70 (d, J=8.8 Hz, 1H), 6.34 (d, J=7.2 Hz, 1H), 6.32 (d, J=2.8 Hz, 1H), 6.18 (d, J=2.8 Hz, 1H), 5.59 (t, J=8.0 Hz, 1H), 4.81 (bs, 1H), 3.91 (s, 3H), 3.62 (m, 2H), 3.39 (m, 2H), 3.11 (dd, J=15.3 and 8.0 Hz, 1H), 2.97 (dd, J=15.3 and 8.0 Hz, 1H), 2.68 (t, J=6.4 Hz, 2H), 2.55 (t, J=7.6 Hz, 2H), 2.01 (m, 2H), 1.89 (m, 2H), and 1.35 (s, 9H);

¹³C NMR (101 MHz; CDCl₃) δ 168.8, 163.8, 156.7, 155.7, 152.4, 145.3, 137.9, 136.8, 127.8, 113.5, 111.4, 111.0, 110.9, 107.6, 81.4, 53.5, 51.5, 43.0, 41.6, 39.8, 34.5, 29.3, 27.9, 26.3, and 21.4.

### Method B:

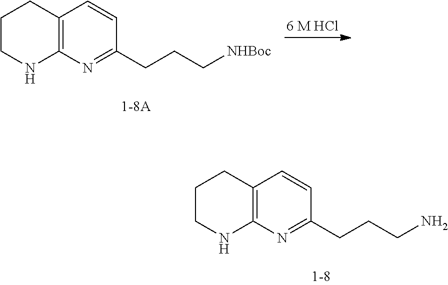 [SMILES: CC(C)(C)OC(=O)NCCCc1ccc2c(n1)NCCC2 ; NCCCc1ccc2c(n1)NCCC2]

To compound 1-8A (for the preparation of 1-9, see U.S. Pat. No. 6,048,861) (10.4 g; 35 mmol) was added 6 M HCl (18 mL) under ice-cooling. The resulting solution was warmed to 35° C. for 1.5 hours. The pH of the solution was adjusted to about 7 with 50 wt % NaOH (˜2 mL) at ambient temperature. After addition of 2-butanol (35 mL) to the mixture, the pH of the aqueous layer was further adjusted to about 11.5 with 50 wt % of NaOH (˜2 mL). The organic layer was separated, washed with saturated aqueous NaCl (10 mL), and dried by distillation at constant volume to remove water to yield a solution of 1-8 in 2-butanol.

A solution of 1-14 (10.0 g; 29 mmol) and triethylamine (5.5 mL; 40 mmol) in THF (45 mL) was added to a solution of bis(trichloromethyl)carbonate (3.51 g; 12 mmol) and THF (75 mL) below 0° C. over 30 minutes. The mixture was aged for 2 hours at ambient temperature. To the mixture was added the 2-butanol solution of 1-8, prepared above, and triethylamine (5.5 mL; 40 mmol). The mixture was aged at 45° C. for 3 hours. To the mixture was added water (20 mL). The organic layer was separated. To the organic layer was added 2 M sulfuric acid (40 mL) and the mixture was aged for 18 hours at ambient temperature. To the mixture was added iPAc (50 mL) and the organic layer was separated. The organic layer was extracted with 2M sulfuric acid (20 mL). The combined aqueous layers were washed with iPAc (50 mL). To a mixture of the resulting aqueous layer and iPAc (80 mL) was added aqueous sodium hydroxide (5 N; 40 mL) under an ice bath to adjust the pH of the aqueous layer to about 8.3. The organic layer was separated and washed with water (3×45 mL). The solution containing the crude 1-16 (12.0 g; 84%) in iPAc was used in the next step without further purification.

### Step L: Preparation of Compound 1-17

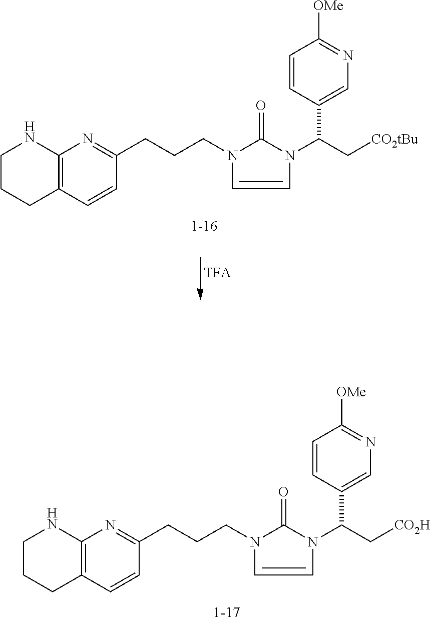 [SMILES: COc1ccc([C@H](CC(=O)OC(C)(C)C)n2ccn(CCCc3ccc4c(n3)NCCC4)c2=O)cn1 ; COc1ccc([C@H](CC(=O)O)n2ccn(CCCc3ccc4c(n3)NCCC4)c2=O)cn1 ; "TFA"]

### Method A:

To a solution of the t-butyl ester (1-16; 37.1 wt % in iPAc; 50 g; 18.6 g as corrected; 0.101 mol) and anisole (21.9 g) was slowly added trifluoroacetic acid (462 g) at 2-3° C. The resulting mixture was stirred at room temperature until reaction completion (4.5 h). Trifluoroacetic acid was removed under vacuum. Isopropyl acetate (100 mL) was added and the solvents removed under vacuum. The flask was cooled with ice and 170 mL of iPAc was added followed by the slow addition of saturated aqueous NH₄OH (170 mL) until pH=10.4. The aqueous layer was separated, washed with 300 mL of iPAc, and concentrated under vacuum until pH=6.5. The resulting solution was subjected to a resin column (Amberchrome CG-161C, Toso-Haas) and first eluted with water to remove trifluoroacetic acid. Subsequently, 50% acetone/water was used to elute the desired product. The fractions containing the product were combined, concentrated in vacuo, and aged at 5° C. The resulting solids were filtered and washed with cold water to give 37.5 g of carboxylic acid 1-17 (85%). Compound 1-17 can be recrystallized from aqueous alcohols, such as methanol, ethanol, or isopropanol, or aqueous acetone.

¹H NMR (400 MHz; CD₃OD): δ 8.16 (d, J=2.6 Hz, 1H), 7.73 (dd, J=8.6 and 2.6 Hz, 1H), 7.45 (d, J=7.4 Hz, 1H), 6.81 (d, J=8.6 Hz, 1H), 6.54 (d, J=3.1 Hz, 1H), 6.53 (d, J=7.4 Hz, 1H), 6.50 (d, J=3.1 Hz, 1H), 5.70 (dd, J=11.6 and 4.2 Hz, 1H), 3.90 (s, 3H), 3.76 (ddd, J=14.1, 9.7 and 4.2 Hz, 1H), 3.51 (dt, J=14.1 and 5.0 Hz, 1H), 3.46 (m, 2H), 2.99 (dd, J=14.0 and 11.6 Hz, 1H), 2.85 (dd, J=14.0 and 4.2 Hz, 1H), 2.77 (t, J=6.4 Hz, 2H), 2.70 (ddd, J=13.8, 8.2 and 6.0 Hz, 1H), 2.50 (dt, J=13.8 and 8.0 Hz, 1H), and 2.16-1.85 (m, 4H);

¹³C NMR (101 MHz, CD₃OD): δ 177.6, 163.9, 153.8, 152.2, 148.8, 145.0, 140.1, 137.9, 128.6, 118.2, 111.1, 110.4, 109.5, 108.6, 52.7, 52.1, 41.5, 40.8, 40.3, 28.9, 28.1, 25.1, and 19.4.

### Method B:

To a solution of 1-16 (140 mg/mL; 220 mL; 30.8 g; 62.4 mmol) in iPAc was added aqueous sulfuric acid (3.06 M; 150 mL), maintaining the batch temperature below 10° C. The aqueous layer was separated and aged at 40° C. for 3 hours. The solution was cooled to 10° C. The pH of the solution was adjusted to about 2 with 50 wt % sodium hydroxide and added SP207 resin (310 mL). The pH of the resulting suspension was adjusted to about 5.9 with 50 wt % sodium hydroxide, and the resulting suspension was aged at ambient temperature for 4 hours. The suspension was filtered and the resin was washed with 930 mL of water. The resin was washed with 70% of acetone-water (v/v; 1.5 L). The fractions containing the product were combined and concentrated to remove acetone. The resulting suspension was cooled to 5° C. The product was collected by filtration and washed with 20 mL of cold water. The crystals were dried at 30° C. under vacuum to give 1-17 (23.5 g; 86% yield).

### Method C:

A solution of 1-16 in iPAc (9.5 g 19.2 mmol; 110 mL) was extracted with aqueous sulfuric acid (3M; 47.5 mL). The aqueous layer was separated and stirred at 40° C. for 3 hours under nitrogen until hydrolysis was completed. The mixture was cooled to about 5° C. and the pH was adjusted to about 1 with aqueous sodium hydroxide (50 wt %). To the mixture was added methanol (71.3 mL). The pH was further adjusted to about 5.0 with aqueous sodium hydroxide (50 wt %) and additional methanol (71.3 mL) was added. The pH was finally adjusted to about 5.9 with aqueous sodium hydroxide (50 wt %). The suspension was stirred at ambient temperature for 1 hour and the resulting salt was filtered and washed with methanol (2×20 mL). The combined filtrate and washings were concentrated and flushed with isopropanol to remove methanol and water. The resulting suspension was stirred at 60° C. to obtain a homogeneous solution. The solution was slowly cooled to 5° C. The suspension was filtered, washed with cold isopropanol (20 mL), and dried under reduced pressure to give colorless crystalline 1-17 (8.1 g; 94 wt %; 91%).

### Step M: Preparation of Compound 1-18

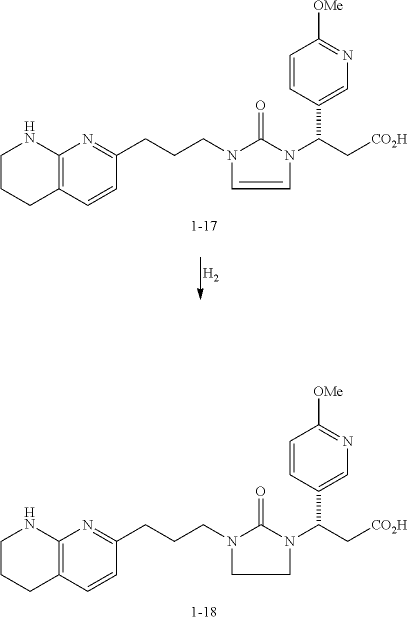 [SMILES: COc1ccc([C@H](CC(=O)O)n2ccn(CCCc3ccc4c(n3)NCCC4)c2=O)cn1 ; "H2" ; COc1ccc([C@H](CC(=O)O)N2CCN(CCCc3ccc4c(n3)NCCC4)C2=O)cn1]

A suspension of 1-17 (105 g), water (247 mL), 5 M NaOH (84 mL) and 20% Pd(OH)₂/C (21 g) was hydrogenated at 120 psi H₂ and 80° C. for 18 h. The pH was adjusted to 9.0 by addition of concentrated HCl (18 mL). The solids were removed by filtration through a pad of Solka Floc® (13 g) and the pad was rinsed with 200 mL of water. The pH of the aqueous solution was adjusted to 6.4 by addition of concentrated HCl and the solution was seeded and aged at 0° C. for 1 h. The solids were collected by filtration and dried under dry nitrogen at room temperature for up to 24 hours to provide 84.5 g (80%) of 1-18 as a colorless crystalline solid. 1-18 is a highly crystalline compound, formed by the process of the present invention in >99.5% enantiomeric excess and >99.5% chemical purity as determined by high-performance liquid chromatography. The 300 MHz NMR spectrum in CD₃OD was identical to that disclosed in U.S. Pat. No. 6,017,926.

The crystalline form obtained was characterized by a differential scanning calorimetry curve, at a heating rate of 10° C./min.under nitrogen, exhibiting a minor endotherm with a peak temperature of about 61° C. due to solvent loss and a major melting endotherm with a peak temperature of about 122° C. (extrapolated onset temperature of about 110° C.). The X-ray powder diffraction showed absorption bands at spectral d-spacings of 3.5, 3.7, 4.3, 5.0, 5.7, 7.1, and 7.5 angstroms. The FT-IR spectrum (in KBr) showed absorption bands at 2922, 2854, 1691, 1495, 1460, 1377, 1288, 1264, and 723 cm⁻¹.

The content of water as obtained with Karl-Fischer titration was 1.7 wt % (the theory for a hemihydrate is 2.0%).

By using appropriate starting materials, Compounds B and D can be synthesized using procedures similar to those described for the synthesis of Compound A.

### Example 2

### Synthesis of Compound C (2-18)

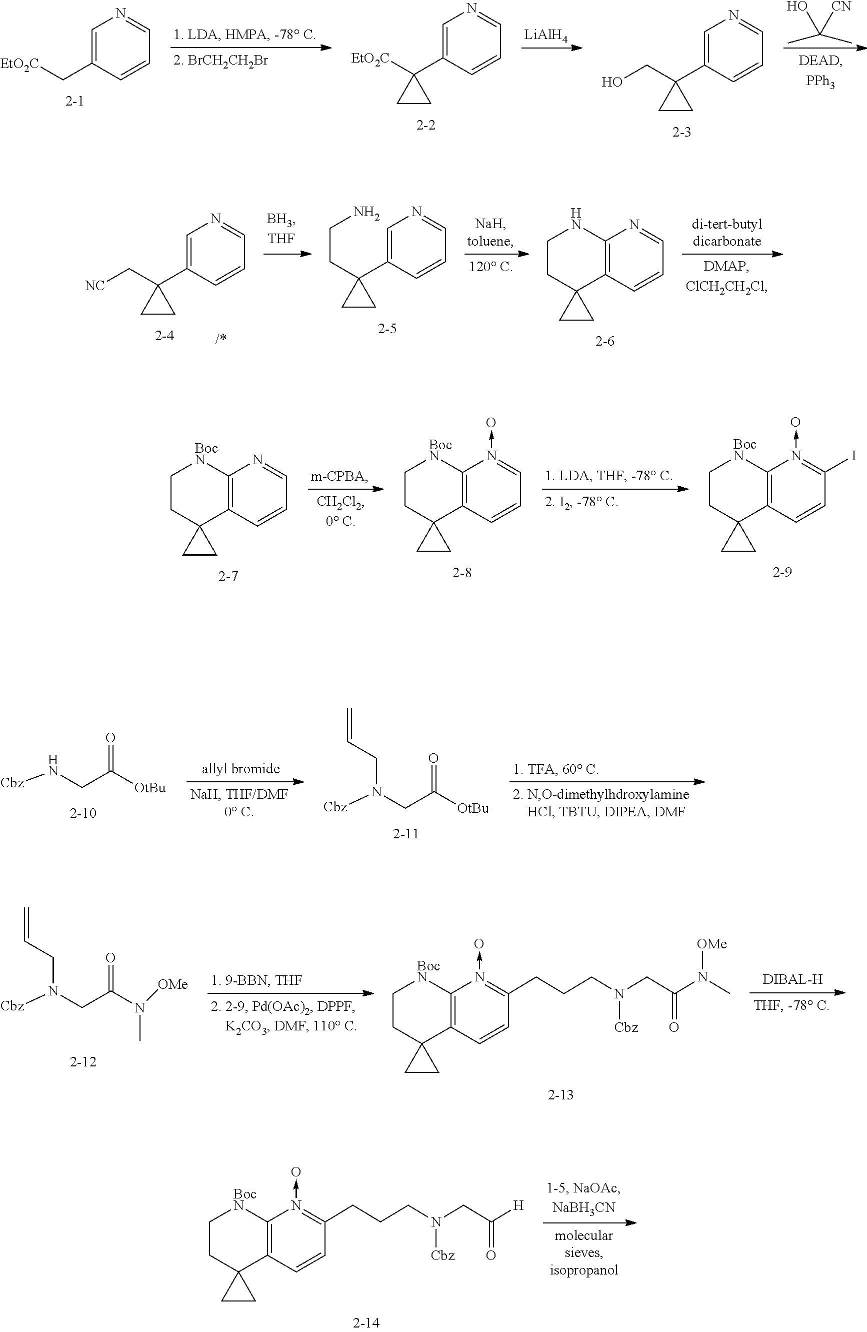

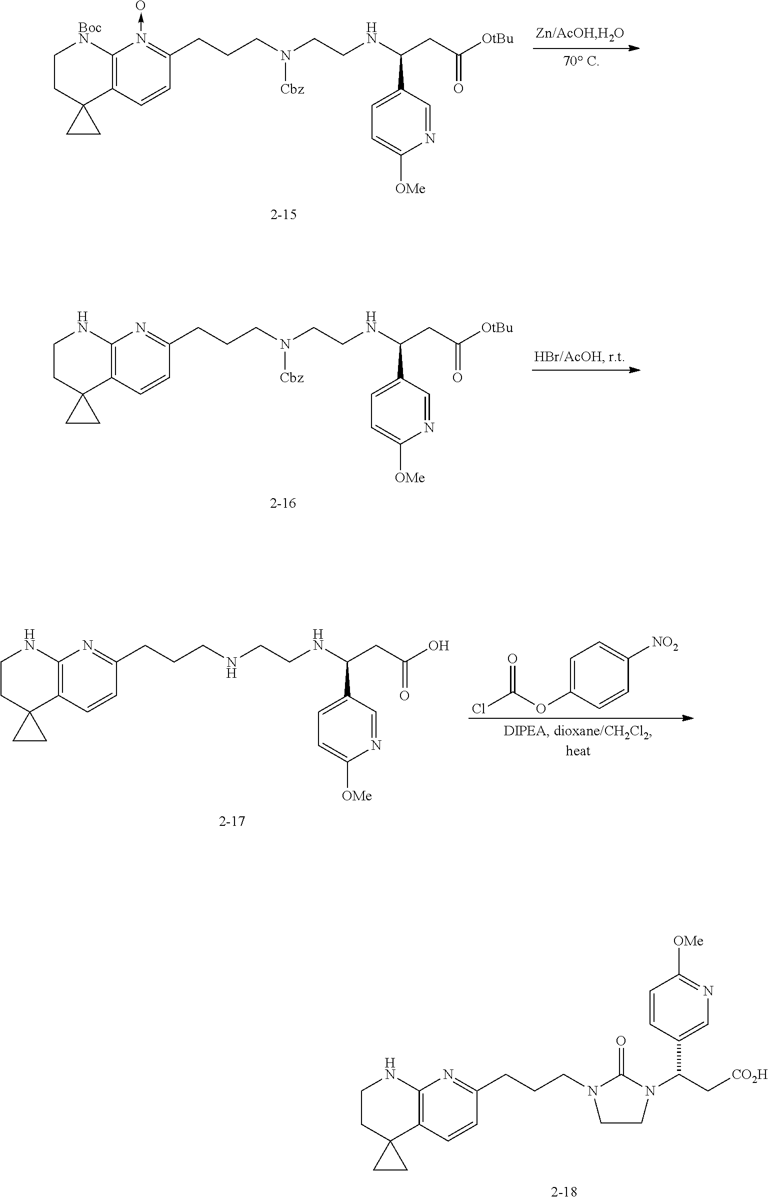 [SMILES: COc1ccc([C@H](CC(=O)OC(C)(C)C)NCCN(CCCc2ccc3c([n+]2[O-])N(C(=O)OC(C)(C)C)CCC32CC2)C(=O)OCc2ccccc2)cn1 ; COc1ccc([C@H](CC(=O)OC(C)(C)C)NCCN(CCCc2ccc3c(n2)NCCC32CC2)C(=O)OCc2ccccc2)cn1 ; COc1ccc([C@H](CC(=O)O)NCCNCCCc2ccc3c(n2)NCCC32CC2)cn1 ; O=C(Cl)Oc1ccc([N+](=O)[O-])cc1 ; COc1ccc([C@H](CC(=O)O)N2CCN(CCCc3ccc4c(n3)NCCC43CC3)C2=O)cn1]

### 1-Pyridin-3-yl-cyclopropanecarboxylic acid methyl ester (2-2)

To a cooled (−78° C.) solution of LDA (2.0 M, 272 mL) in 500 mL anhydrous THF and 200 mL HMPA (dried with molecular sieves) was added gradually a solution of ethyl 3-pyridylacetate 2-1 (75.0 g, 454 mmol) in 50 mL THF. The mixture was stirred for 50 min at −78° C. and treated with neat 1,2-dibromoethane (117 mL, 1363 mmol) in one portion. The reaction mixture was stirred overnight while being allowed to warm to room temperature. The reaction mixture was quenched with saturated NH₄Cl and extracted three times with EtOAc. The combined organic layers were washed three times with H₂O and then brine. After solvent removal, the residue was purified using silica gel chromatography (100% hexanes to EtOAc/hexane=7/3) to obtain the desired product 2-2 as an oil.

¹H NMR (400 MHz, CDCl₃): δ 8.60 (m, 1H), 8.50 (m, 1H), 7.64 (m, 1H), 7.28 (m, 1H), 4.05 (q, 2H), 1.66 (q, 2H), 1.22 (q, 2H), 1.16 (t, 3H).

### (1-Pyridin-3-yl-cyclopropyl)-methanol (2-3)

To a cooled (−78° C.) solution of 2-2 (76 g, 398 mmol) in 500 mL THF was added LiAlH₄ (1.0 M, 250 mL, 250 mmol) gradually. The reaction mixture was stirred for 2 hr and quenched sequentially with 9.5 mL H₂O, 9.5 mL 15% NaOH, and 28.5 mL H₂O. The mixture was stirred overnight. Celite (50 g) was added and the mixture stirred for 20 min and filtered through a silica gel plug and concentrated to afford the desired product 2-3 as an oil which was used in the next step without further purification.

¹H NMR (400 MHz, CDCl₃): δ 8.58 (m, 1H), 8.40 (m, 1H), 7.65 (m, 1H), 7.20 (m, 1H), 3.70 (s, 2H), 0.90 (m, 4H).

### (1-Pyridin-3-yl-cyclopropyl)-acetonitrile (2-4)

To a cooled (−20° C.) solution of PPh₃ (2.6 g, 10 mmol) in 30 mL ether was added over 5 minutes a solution of DEAD (1.8 g, 10 mmol) in 20 mL ether. The mixture was stirred for 25 min at −20° C. A solution of 2-3 (1.0 g, 6.7 mmol) in 10 ml ether was added, and the reaction mixture was stirred for 30 min at −20° C. Acetone cyanohydrin (1.9 g, 20 mmol) was then added. The reaction mixture was stirred overnight while being allowed to warm to room temperature. After solvent removal, the residue was purified using silica gel chromatography (100% hexanes to 100% EtOAc) to obtain the desired product 2-4 as an oil.

¹H NMR (400 MHz, CDCl₃): δ 8.65 (m, 1H), 8.54 (m, 1H), 7.72 (m, 1H), 7.29 (m, 1H), 2.68 (s, 2H), 1.06 (s, 4H).

### 2-(1-Pyridin-3-yl-cyclopropyl)-ethylamine (2-5)

To a cooled (0° C.) solution of 2-4 (31.3 g, 198 mmol) in 200 mL anhydrous THF was added borane-THF solution (1.5 M, 660 mL, 990 mol). The reaction mixture was stirred at room temperature overnight. It was then quenched with methanol gradually until no gas was released. Then additional methanol (150 ml) was added, followed by 30 mL of 6N HCl. The mixture was stirred for 1 hr and concentrated to a viscous residue. It was then treated with 6N NaOH until pH >11 and stirred for 30 min and extracted four times with CHCl₃. After solvent removal, the residue was purified using silica gel chromatography (100% EtOAc to 50% EtOAc/46% EtOH/2% NH₄OH/2% H₂O) to obtain the desired product 2-5 as an oil.

Mass spectrum: Observed for [M+H]⁺ 163.2; Calculated 162.12.

### 1,2,3,4-Tetrahydro-4,4-ethyleno-[1,8]naphthyridine (2-6)

To a mixture of 2-5 (15.0 g, 92.6 mmol) and 300 mL anhydrous toluene was added NaH (17.8 g, 445 mmol) gradually under nitrogen. The suspension was stirred at 120° C. for 8 hr. It was then cooled and quenched very slowly with EtOH until it became homogeneous. 150 mL of saturated NaHCO₃ was added. The mixture was extracted three times with EtOAc. The combined organic layers were washed with brine and dried (MgSO₄). After solvent removal, the residue was purified using silica gel chromatography (EtOAc/hexanes=1:2 to 100% EtOAc) to obtain the desired product 2-6 as an oil.

Mass spectrum: observed for [M+H]⁺161.1; Calculated 160.12.

### 1,2,3,4-Tetrahydro-4,4-ethyleno-[1,8]naphthyridine-1-carboxylic acid tert-butyl ester (2-7)

A mixture of 2-6 (8.50 g, 53.1 mmol), di-tert-butyl dicarbonate (34.8 g, 159 mmol), and DMAP (0.13 g, 1.06 mmol) in 70 ml of 1,2-dichloroethane was heated at reflux for 5 hours, then cooled to room temperature, washed with saturated Na₂CO₃ solution, and brine separately. The solvents were removed under reduced pressure and the residue was purified by flash silica gel column chromatography (100% hexanes to 100% EtOAc) to provide 2-7 as a pale solid.

¹H NMR (400 MHz, CDCl₃): δ 8.28 (m, 1H), 6.95 (m, 2H), 3.91 (m, 2H), 1.81 (m, 2H), 1.55 (s, 9H), 1.01 (m, 2H), 0.92 (m, 2H).

### 8-Hydroxy-1,2,3,4-tetrahydro-4,4-ethyleno-[1,8]naphthyridine-1-carboxylic acid tert-butyl ester (2-8)

The mixture of 2-7 (5.85 g, 22.5 mmol) and 3-chloroperoxybenzoic acid (mCPBA) (6.11 g, 24.8 mmol) in 100 ml of CH₂Cl₂ was stirred at room temperature for three hours. The solvent was removed, the residue was diluted with water, and extracted with EtOAc. The combined organic extracts were washed with brine, dried with Na₂SO₄. After the solvent was removed, pure product 2-8 was obtained via flash silica gel column chromatography (100% EtOAc to 10% MeOH/90% EtOAc).

¹H NMR (400 MHz, CDCl₃): δ 7.98 (m, 1H), 6.86 (m, 1H), 6.50 (m, 1H), 3.95 (m, 1H), 3.61 (m, 1H), 1.75 (m, 2H), 1.44 (s, 9H), 1.03 (m, 2H), 0.95 (m, 2H).

### 8-Hydroxy-7-iodo-1,2,3,4-tetrahydro-4,4-ethyleno-[1,8]naphthyridine-1-carboxylic acid tert-butyl ester (2-9)

A solution of 2-8 (5.40 g, 19.5 mmol) in 50 ml of THF was added to a solution of LDA (2.0 M in heptanes, THF, and ethylbenzene, 11.8 ml, 23.5 mmol) in 100 ml of THF at −78° C. under nitrogen. After the mixture was stirred at −78° C. for 1 hour, a solution of iodine (9.90 g, 39.0 mmol) in 50 ml of THF was added via cannula. The resulting mixture was stirred at −78° C. for 90 minutes, then quenched with AcOH (2.6 ml), warmed to room temperature and diluted with H₂O, NaHCO₃ (aq), and Na₂S₂O₃ (aq). The mixture was extracted with EtOAc, the combined organic extracts were washed with brine, and dried with MgSO₄. After the solvent was removed, the product 2-9 was purified by flash silica gel column chromatography (20% EtOAc/80% hexanes to 65% EtOAc/35% hexanes).

¹H NMR (400 MHz, CDCl₃): δ 7.53 (d, 1H), 6.24 (d, 1H), 4.13 (m, 1H), 3.52 (m, 1H), 1.95 (m, 1H), 1.54 (m, 1H), 1.49 (s, 9H), 1.05 (m, 4H).

### (Allyl-benzyloxycarbonyl-amino)-acetic acid tert-butyl ester (2-11)

To a mixture of 2-10 (6.30 g, 23.7 mmol) and allyl bromide (filtered through a short plug of poly(4-vinylpyridine) before use, 2.40 ml, 26.1 mmol) in 50 ml of THF and 50 ml of DMF at 0° C. under nitrogen was added NaH (60% dispersion in mineral oil, 1.05 g, 26.1 mmol) in one portion. The resulting mixture was stirred at 0° C. for 30 minutes, then at room temperature for three hours, quenched with saturated aqueous NH₄Cl solution, diluted with H₂O, and extracted with EtOAc. The combined organic extracts were washed with H₂O, brine, and dried with MgSO₄. After the solvent was removed, the product was purified by flash silica gel column chromatography (0% to 20% of EtOAc in hexanes).

¹H NMR (400 MHz, CDCl₃): δ 7.34 (m, 5H), 5.80 (m, H), 5.16 (m, 4H), 4.00 (m, 2H), 3.87 (m, 2H), 1.41 (m, 9H).

### Allyl-[(methoxy-methyl-carbamoyl)-methyl]-carbamic acid benzyl ester (2-12)

A mixture of 2-11 (6.0 g, 19.6 mmol) and 10 ml of TFA was stirred at 60° C. for 20 minutes. The volatiles were removed under reduced pressure, the residue was azeotroped with toluene (20 ml×3), then dissolved in 60 ml of anhydrous DMF, and to which was added N,O-dimethylhydroxylamine hydrochloride (2.20 g, 21.7 mmol), DIPEA (10.3 ml, 59.1 mmol), and TBTU (6.97 g, 21.7 mmol) at room temperature. The resulting mixture was stirred for 1.5 hr, diluted with H₂O, and extracted with EtOAc. The organic layer was then washed with saturated aqueous Na₂CO₃ solution, H₂O, and brine separately, and then dried with MgSO₄. After the solvent was removed, the product was purified by flash silica gel column chromatography (0% to 45% of EtOAc in hexanes).

¹H NMR (400 MHz, CDCl₃): δ 7.34 (m, 5H), 5.81 (m, H), 5.17 (m, 4H), 4.10 (m, 4H), 3.73/3.54 (s, 3H), 3.20/3.15 (s, 3H).

### 1-Hydroxy-2-(3-{benzyloxycarbonyl-[(methoxy-methyl-carbamoyl)-methyl]-amino}-propyl)-8-tert-butoxycarbonyl-5,6,7,8-tetrahydro-5,5-ethyleno-[1,8]naphthyridine (2-13)

A mixture of 2-12 (4.1 g, 14.0 mmol) and 9-BBN (0.5 M solution in THF, 34.0 ml, 16.8 mmol) was stirred at room temperature under nitrogen for 15 hours. The volatiles were removed under reduced pressure, the residue was dissolved in 150 ml of DMF, and to which was added 2-9 (5.55 g, 13.8 mmol), K₂CO₃ (2.90 g, 21.0 mmol), Pd(OAc)₂ (0.31 g, 1.40 mmol), and DPPF (0.78 g, 1.40 mmol). The resulting mixture was then stirred at 60° C. for 1 hour, at 110° C. for 30 minutes, cooled to room temperature, diluted with H₂O, and extracted with EtOAc. The combined organic extracts were washed with H₂O, brine, and dried with MgSO₄. After the solvents were removed, the product was purified by flash silica gel column chromatography (0% to 80% of EtOAc/MeOH (8:2) in hexanes).

¹H NMR (400 MHz, CDCl₃): δ 7.34 (m, 5H), 5.81 (m, H), 5.17 (m, 4H), 4.10 (m, 4H), 3.73/3.54 (s, 3H), 3.20/3.15 (s, 3H). MS: [M+H]⁺=569.1

### 1-Hydroxy-2-[3-(benzyloxycarbonyl-{2-[1-(6-methoxy-pyridin-3-yl)-3-oxo-butylamino]-ethyl}-amino)-propyl)]-8-tert-butoxycarbonyl-5,6,7,8-tetrahydro-5,5-ethyleno-[1,8]naphthyridine (2-15)

DIBAL-H (1.0 M solution in hexanes, 8.80 ml, 8.80 mmol) was added dropwise to a stirred solution of 2-13 (2.00 g, 3.52 mmol) in 40 ml of anhydrous THF at −78° C. After 2 hours, the mixture was warmed to room temperature and quenched by slow addition of MeOH (1.6 ml). A 1.0 M aqueous Rochelle salt solution was added, and the mixture was stirred for 30 minutes. EtOAc was added, the organic layer was separated and dried with MgSO₄, the solvent was removed under reduced pressure, and the crude product 2-14 was azeotroped with toluene, then dissolved in 40 ml of isopropanol. To the solution was added 3(S)-(6-methoxypyridin-3-yl)-β-alanine tert-butyl ester p-toluenesulfonic acid salt 1-5 (1.73 g, 4.22 mmol), NaOAc (2.89 g, 35.2 mmol), and 3.5 g of powdered molecular sieves. The mixture was stirred at room temperature for 12 hours and was cooled to 0° C., and NaCNBH₃ (0.67 g, 10.6 mmol) was added in one portion. The mixture was then warmed to room temperature and stirred for 24 hours. 1H HCl was added to bring the pH to 2. After the mixture was stirred for 10 minutes, EtOAc was added, the pH was then adjusted to 11 with saturated aqueous Na₂CO₃. The organic portion was separated and dried with MgSO₄, filtered, concentrated, and purified by flash silica gel column chromatography (0% to 10% of MeOH in EtOAc).

Mass spectrum: Observed [M+H]⁺=746.3.

### 3-(2-{Benzyloxycarbonyl-[3-(5,6,7,8-tetrahdro-5,5-ethyleno-[1,8]naphthyridin-2-yl)-propyl]-amino}-ethylamino)-3(S)-(6-methoxypyridin-3-yl)-propionic acid tert-butyl ester (2-16)

Zinc powder (100 mesh, 2.0 g, 30.2 mmol) was added in one portion to the solution of 2-15 (1.50 g, 2.01 mmol) in 12 ml of AcOH and 2 ml of H₂O at 70° C. The mixture was then stirred at 70° C. for 30 minutes and then cooled to room temperature. The solids were removed by filtration, the solvents were removed under reduced pressure, and the residue was partitioned between EtOAc and 5% aqueous NH₄OH. The organic layer was then washed with brine and dried with MgSO₄. The solvent was removed to afford the crude product which was used in the preparation of 2-17 without further purification.

Mass spectrum: observed [M+H]⁺=630.2.

### 3(S)-(6-Methoxy-pyridin-3-yl)-3-{2-[3-(5,6,7,8-tetrahydro-5,5-ethyleno-[1,8]naphthyridin-2-yl)-propylamino]-ethylamino}-propionic acid (2-17)

Crude 2-16 (2.01 mmol) in 3 ml of HBr (30 wt. % solution in AcOH) and 3 ml of AcOH was stirred at room temperature for 30 minutes, ether was added, the mixture was stirred for 10 minutes, the ether solution was removed by decantation, the residue was purified by flash silica gel column chromatography

(5% to 20% of MeOH in CH₂Cl₂ with 4% of NH₄OH) to afford the title compound. Mass spectrum: observed [M+H]⁺=440.2.

### 3(S)-(6-Methoxy-pyridin-3-yl)-3-{2-oxo-3-(5,6,7,8-tetrahydro-5,5-ethyleno-[1,8]naphthyridin-2-yl)-propyl]imidazolidin-1-yl}-propionic acid (2-18)

A solution of 4-nitrophenyl chloroformate (0.29 g, 1.46 mmol) in 20 ml of 1,4-dioxane was added dropwise to a mixture of 2-17 (0.61 g, 1.39 mmol) and DIPEA (1.1 ml, 6.26 mmol) in 150 ml of 1,4-dioxane and 60 ml of CH₂Cl₂ at 0° C. under nitrogen. The resulting mixture was stirred at 0° C. for 40 minutes, warmed to room temperature, then heated at reflux for three hours. The volatiles were removed under reduced pressure and the product was purified by flash silica gel column chromatography (5% to 15% of MeOH in CH₂Cl₂ with 3% of NH₄OH).

¹H NMR (400 MHz): δ 11.0 (s, broad, 1H), 8.11 (m, 1H), 7.57 (m, 1H), 6.91 (d, 1H), 6.72 (d, 1H), 6.28 (d, 1H), 5.57 (m, 1H), 3.92 (s, 3H), 3.38-3.66 (m, 5H), 3.16 (q, 1H), 2.95-3.03 (m, 2H), 2.65-2.85 (m, 4H), 1.90-1.98 (m, 1H), 1.74-1.83 (m, 1H), 1.69 (t, 2H), 1.01 (m, 2H), 0.83 (m, 2H).

Mass spectrum: [M+H]⁺=466.2.

### Example 3

### Synthesis of Compound E (3-8)

### Step A: 1-(Pyrimidin-5-yl)-7-(5,6,7,8-tetrahydro-[1,8]naphthyridin-2-yl)-hept-1-en-3-one (3-2)

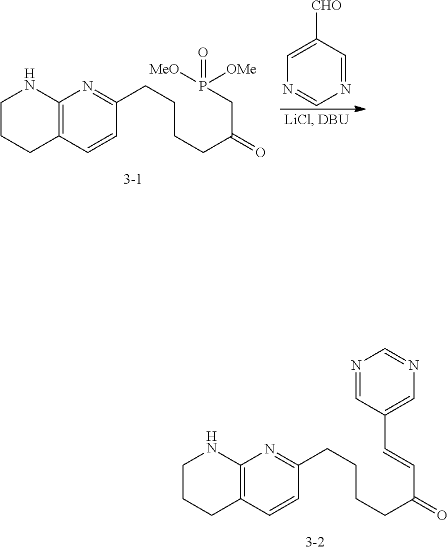 [SMILES: COP(=O)(CC(=O)CCCCc1ccc2c(n1)NCCC2)OC ; O=Cc1cncnc1 ; O=C(/C=C/c1cncnc1)CCCCc1ccc2c(n1)NCCC2]

To a stirred suspension of anhydrous lithium chloride (3.54 g, 83.3 mmol) in acetonitrile (350 mL) at room temperature was added a solution of ketophosphonate 3-1 (for preparation of 3-1, see U.S. Pat. No. 6,048,861) (28.3 g, 83.1 mmol) in acetonitrile (128 mL). After stirring for 15 min, a solution of DBU (9.52 mL, 64.1 mmol) in acetonitrile (32 mL) was added to produce a mostly fine white precipitate with some larger masses. The reaction mixture was briefly sonicated to break up the larger masses and stirred for 30 min. A solution of pyrimidine-5-carboxaldehyde (6.92 g, 64.1 mmol) in acetonitrile (128 mL) was added over 15 min. After 2 h, the reaction mixture was filtered and the filtrate concentrated. The residue was purified by flash chromatography (8% MeOH/EtOAc) to give 18.5 g (90%) of enone 3-2 as a yellow crystalline solid; m.p. 101-102° C.

¹H NMR (399.87 MHz, CDCl₃): δ 9.19 (s, 1H), 8.89 (s, 2H), 7.45 (d, J=16.3 Hz, 1H), 7.05 (d, J=7.3 Hz, 1H), 6.85 (d, J=16.3 Hz, 1H), 6.35 (d, J=7.3 Hz, 1H) 4.78 (br s, 1H), 3.39 (m, 2H), 2.72-2.67 (om, 4H), 2.58 (m, 2H), 1.89 (m, 2H), 1.79-1.72 (om, 4H) ppm.

¹³C NMR (100.55 MHz, CDCl₃): δ 199.3, 159.40, 159.36, 158.0, 155.9, 136.8, 134.7, 129.4, 128.8, 113.5, 111.5, 41.8, 41.6, 37.7, 29.5, 26.5, 23.9, 21.7 ppm.

### Step B: Preparation of the “Modified” (R)-BINAL-H Reagent

To a dry 500 mL 3-neck round bottom flask at room temperature was added dry toluene (25 mL) followed by LAH (1.76 g, 46.4 mmol) under a nitrogen atmosphere. The resulting gray slurry mixture was treated with THF (7.2 mL), which was added over 10 min. at temperature <30° C. The resulting mixture was heated to 35° C. and treated with a solution of ethanol in toluene (6 M, 7.5 mL, prepared by adding 2.5 mL of ethanol in 4.9 mL of toluene), which was added slowly over 30 minutes between 35 and 40° C. After complete addition, the slurry was aged at 35° C. for 40 minutes and then cooled to 30° C. The resulting mixture was then treated with a solution of (R)-(+)-BINOL (12.3 g, 46 mmol) in toluene (90 mL) at 30° C., which was added at such a rate such that the batch temperature was maintained at <40° C., with cooling in an ice-bath if necessary. The resulting light gray slurry mixture was heated to 50° C. and aged for 1 hour and then allowed to cool to room temperature. The light gray mixture was then heated back up to 50° C. and treated with TMEDA (20.2 mL, 134 mmol) and stirred at 50° C. for 1 hour and then allowed to cool to room temperature. The total volume was 164 mL or ˜0.27 M solution of “modified” (R)-BINAL-H in toluene/THF solution. The solution was used directly in the following reduction step C without further purification.

### Step C: (R)-1-(Pyrimidin-5-yl)-7-(5,6,7,8-tetrahydro-[1,8]-naphthyridin-2-yl)-(E)-hept-1-en-3-ol (3-3)

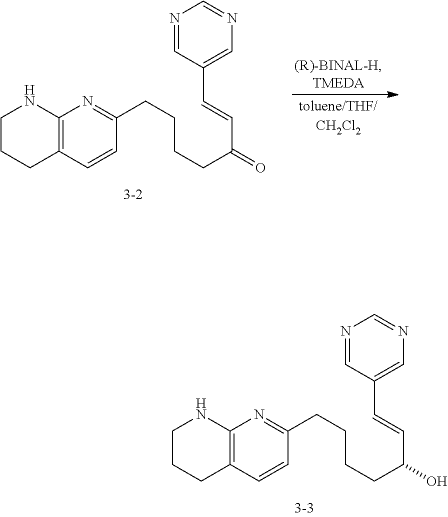 [SMILES: O=C(/C=C/c1cncnc1)CCCCc1ccc2c(n1)NCCC2 ; O[C@@H](/C=C/c1cncnc1)CCCCc1ccc2c(n1)NCCC2]

To a dry 500 mL 3-neck round bottom flask was added a toluene/THF solution of “modified” (R)-BINAL-H from Step B (0.27 M, 120 mL, 3.2 equiv.) under a nitrogen atmosphere, and the solution was cooled to −75 to −73° C. with a dry-ice acetone bath. Then a solution of enone 3-2 (3.3 g, 10.2 mmol) in DCM (23 mL) was added over 45 minutes while maintaining the batch temperature between −73 to −69° C. The reaction mixture was aged at −75° C. to −70° C. for 40 minutes and quenched with methanol (4 mL, 102 mmol) at −70° C. and then allowed to warm to room temperature. The reaction mixture was monitored by chiral HPLC: Chiralpak AD Analytical Column, 4.6×250 mm, 5 micron pore size; mobile phase: ethanol (with 0.1 v/v % diethylamine); flow rate: 2.0 mL/min.; injection volume=10 μL; detection=250 nm, sample preparation=100× dilution. Approximate retention times were:

| retention time (min.) | identity |
|---|---|
| 5.8 | (R)-allylic alcohol 3 |
| 6.9 | (S)-allylic alcohol 3 |
| 10.8 | enone 2 |

The reaction was deemed complete when the enone was <1.0 area %. The optical purity of (R)-3-3 was ˜80% enantiomeric excess (ee).

The reaction mixture was filtered through a pad of Solka Floc® and the pad rinsed with DCM (20 mL). The resulting filtrate was transferred to a separatory funnel and extracted twice with aqueous tartaric acid solution (2.0 M, 1×100 mL and 1×50 mL). The combined aqueous phase was washed with DCM (20 mL). The pH of the washed aqueous phase was adjusted to 7 to 8 with 23 wt. % aqueous ammonium hydroxide solution and extracted with DCM (3×60 mL). The combined DCM solution was washed with 0.5 M ammonium chloride solution (3×100 mL) and dried over sodium sulfate. The solution was filtered and concentrated under reduced pressure to an oil. The resulting residue was dissolved in acetonitrile (100 mL) and concentrated to 10% of the initial volume and treated with additional acetonitrile (90 mL) and concentrated back to an oily residue.

The resulting residue (3.0 g) was charged into a 250-mL, 3 neck-round bottom flask, which was equipped with a temperature probe, a nitrogen inlet adapter, a magnetic stirrer, and a heating mantel, and treated with acetonitrile (60 mL) and then heated to 40° C. and aged 15 min. The resulting solution was then allowed to cool to room temperature and stirred overnight at room temperature.

The supernatant was checked by chiral HPLC assay at two wavelengths, 250 and 330 nm. After stirring at room temperature for 3 h, the (R)-allylic alcohol in acetonitrile solution was assayed to be 95% ee for (R)-3-3.

The slurry mixture was then cooled to 10° C. and filtered to isolate the (R)-allylic alcohol 3-3 as an acetonitrile solution (60 mL; 28 g/L; 1.7 g; 52% recovery) in a HPLC area % purity of 70% and in a chiral HPLC purity of 98% ee.

The HPLC purity (area %) was determined by gradient HPLC assay: YMCbasic AD Analytical Column, 4.6×250 mm, 5 micron pore size; Gradient Elution: Solvent A=5.0 mM each KH₂PO₄ and K₂HPO₄, Solvent B=Acetonitrile, T=0 min. A 70% A:30% B. T=20 min. @ 20% A:80% B, T=21 min. @ 70% A:30% B; 1.0 mL/min.; injection volume=10 μL; detection=250 nm; sample preparation=100× dilution. Approximate retention times were:

| retention time (min.) | identity |
|---|---|
| 6.2 | (R)-allylic alcohol 3-3 |
| 7.9 | enone 3-2 |

### Step D: Methyl malonate ester of (R)-1-(pyrimidin-5-yl)-7-(5,6,7,8-tetrahydro-[1,8]-naphthyridin-2-yl)-(E)-hept-1-en-3-ol (3-4)

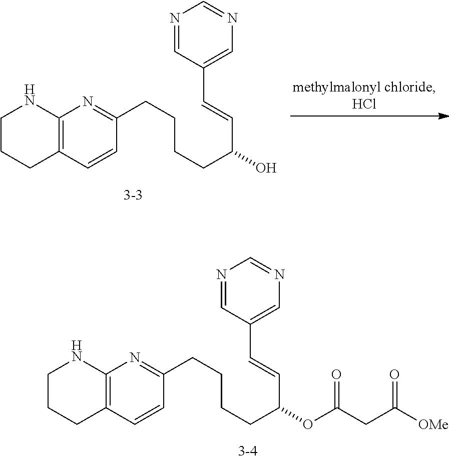 [SMILES: O[C@@H](/C=C/c1cncnc1)CCCCc1ccc2c(n1)NCCC2 ; COC(=O)CC(=O)O[C@@H](/C=C/c1cncnc1)CCCCc1ccc2c(n1)NCCC2]

A 250-mL vessel was charged with allylic alcohol 3-3 (97% ee) (14.9 g) and DCM (90 mL) at 20° C. The solution was cooled to 5° C. To the resulting solution was added 2M hydrogen chloride in isopropyl acetate (prepared by adding HCl gas to isopropyl acetate at 0-20° C. by weight, 22 mL) while maintaining the temperature at 5-10° C. Methylmalonyl chloride (5.7 mL) was next added over 30 min, while maintaining the temperature at 5-10° C. during the addition. The reaction mixture was stirred for 1-2 h at 5° C. Excess methylmalonyl chloride was quenched with methanol (1.5 mL) and the solution stirred for 5 min. 2M Aqueous potassium hydrogencarbonate solution (70 mL) was added over 30 min at 5-10° C. The two-phase mixture was allowed to stir for 20 min at 10-15° C. The lower organic layer was removed and the aqueous layer extracted with DCM (20 mL). The combined organic layers were concentrated to about 20% of the original volume, and DCM (100 mL) was added. The mixture was concentrated to about 30 mL and diluted with NMP (35 mL). The residual DCM was removed under diminished pressure at 10-20° C., and the resulting solution used in Step E below.

### Step E: 3(R)-(Pyrimidin-5-yl)-9-(5,6,7,8-tetrahydro-[1,8]-naphthyridin-2-yl)-(E)-non-4-enoic acid methyl ester (3-5)

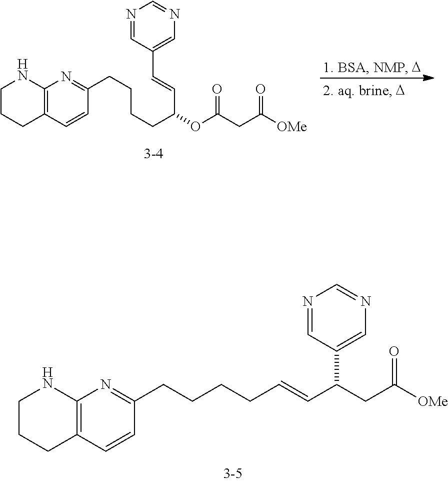 [SMILES: COC(=O)CC(=O)O[C@@H](/C=C/c1cncnc1)CCCCc1ccc2c(n1)NCCC2 ; COC(=O)C[C@H](/C=C/CCCCc1ccc2c(n1)NCCC2)c1cncnc1]

To a solution of the malonate ester 3-4 from Step D in NMP was added BSA (32 mL) at 20° C. and the solution warmed to 60° C. The solution was kept at 60° C. for 30 min. 10% Aqueous brine (4.7 mL) was then added over 20 min. The resulting solution was warmed to 90° C. for 1 h. The reaction mixture was then cooled to 20° C. and washed with heptane (2×30 mL). The NMP layer was recharged to the reaction vessel. Water (150 mL) was added followed by ethyl acetate (75 mL). The two-phase mixture was stirred for 20 min, and the lower aqueous layer separated and reextracted with ethyl acetate (2×50 mL). The organic layers were combined and washed with water (2×30 mL). The organic layers were concentrated to 20% volume.

Chiral purity was assayed to be >97% ee for (R) 3-5 (AD normal phase liquid chromatography).

### Step F: 3(S)-(Pyrimidin-5-yl)-9-(5,6,7,8-tetrahydro-[1,8]-naphthyridin-2-yl)-nonanoic acid methyl ester (3-6)

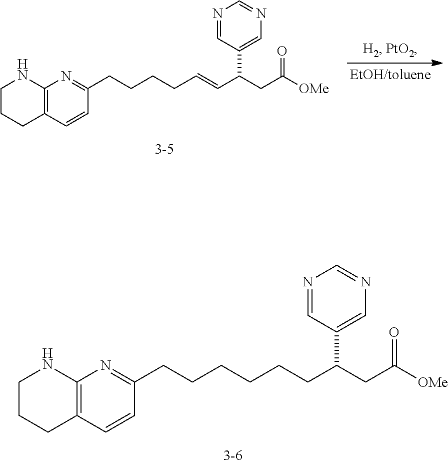 [SMILES: COC(=O)C[C@H](/C=C/CCCCc1ccc2c(n1)NCCC2)c1cncnc1 ; COC(=O)C[C@H](CCCCCCc1ccc2c(n1)NCCC2)c1cncnc1]

To a solution of the unsaturated ester 3-5 (20.0 g) in ethanol (53 mL) was added platinum oxide (0.8 g). Following a series of degas cycles, the mixture was placed under 40 psi of hydrogen gas and heated for 24-36 h at 50° C. The catalyst was removed by filtration through Solka Floc®, and the filtrate evaporated to an oil, which was diluted with toluene (40 mL) and stirred for 15 min. This mixture was filtered over a plug of silica gel (20 g) slurried in toluene (30 mL). The filtrate was collected, and the filter was washed with an additional 250 mL of toluene/ethanol (4:1 by volume). The solvent was switched to toluene to afford the saturated ester 3-6.

The assay yield was 95-97%, 97% ee.

### Step G: 3(S)-(Pyrimidin-5-yl)-9-(5,6,7,8-tetrahydro-[1,8]-naphthyridin-2-yl)-nonanoic acid (3-7)

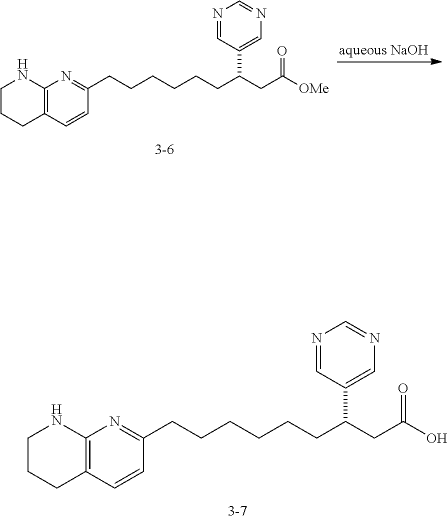 [SMILES: COC(=O)C[C@H](CCCCCCc1ccc2c(n1)NCCC2)c1cncnc1 ; O=C(O)C[C@H](CCCCCCc1ccc2c(n1)NCCC2)c1cncnc1]

To a solution of the saturated ester 3-6 (50 g) in toluene (250 mL), which was filtered through a one micron filter, was added water (120 mL) followed by 50% w/w sodium hydroxide (13.3 g) and an additional charge of water (30 mL). The biphasic mixture was stirred vigorously and heated for 3 h at 50° C. The mixture was cooled and the pH adjusted to 8.0 with 2M phosphoric acid. The aqueous layer was separated and residual toluene removed under vacuum. The mixture was adjusted to pH 7.5 and seeded. After 1 h, the pH was slowly adjusted to 6.0 over 1 h. After stirring overnight, the mixture was filtered and the solid washed with water (2×97 mL). The solid was dried under vacuum. Isolated yield was 92%, 97% ee. The 400 MHz NMR spectrum of 3-7 in methanol-d₄ was identical to that reported for compound 19-3 in U.S. Pat. No. 6,048,861.

### Alternate Method

### Step A: 3(R)-(Pyrimidin-5-yl)-9-(5,6,7,8-tetrahydro-[1,8]-naphthyridin-2-yl)-(E)-non-4-enoic acid (3-8)

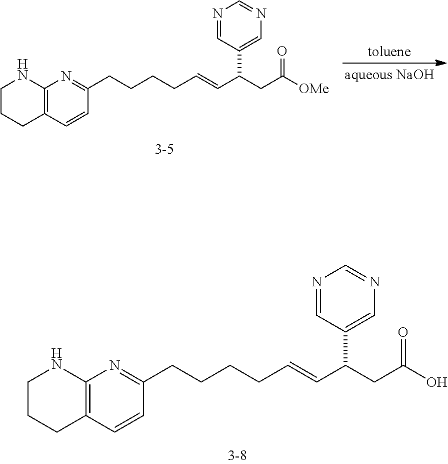 [SMILES: COC(=O)C[C@H](/C=C/CCCCc1ccc2c(n1)NCCC2)c1cncnc1 ; O=C(O)C[C@H](/C=C/CCCCc1ccc2c(n1)NCCC2)c1cncnc1]

To a solution of the unsaturated ester 3-5 (31 g) in toluene (105 mL) was added water (75 mL) followed by 5N aqueous sodium hydroxide (19.5 mL). The biphasic mixture was stirred vigorously and heated for 3 h at 50° C. The mixture was cooled and the pH adjusted to 8.0 with 2M phosphoric acid. The aqueous layer was separated and residual toluene removed under vacuum. The mixture was adjusted to pH 7.5 and seeded. After 1 h, the pH was slowly adjusted to 6.0 over 1 h. After stirring overnight, the mixture was filtered and the solid washed with water (60 mL). The solid was dried on a sintered-glass funnel (nitrogen suction) over 2-3 days. The title compound 3-8 was isolated as an off-white solid in 95% yield.

¹H NMR (400 MHz, CDCl₃): δ 9.03 (s, 1H), 8.62 (s, 2H), 7.14 (d, 1H), 6.21 (d, 1H), 5.66 (m, 1H), 5.53 (m, 1H), 3.85 (m, 1H), 3.39 (m, 2H), 2.68 (m, 5H), 2.53 (m, 1H), 2.10 (m, 1H), 2.02 (m, 1H), 1.90-1.78 (m, 3H), 1.63 (m, 1H), 1.46 (m, 1H), 1.37 (m, 1H).

### Step B: 3(S)-(Pyrimidin-5-yl)-9-(5,6,7,8-tetrahydro-[1,8]-naphthyridin-2-yl)-nonanoic acid (3-7)

To a solution of the unsaturated acid 3-8 (1.2 g) in methanol (2.0 mL) was added platinum oxide (24 mg). Following a series of degas cycles, the mixture was placed under 40 psi of hydrogen gas and stirred for 17 h at 20° C. HPLC assay indicated 99% conversion to the saturated acid 3-7. The catalyst was removed by filtration, and the filtrate evaporated to afford 7 as a solid that was dried under vacuum. The 400 MHz NMR spectrum of 3-7 in methanol-d₄ was identical to that reported for compound 19-3 in U.S. Pat. No. 6,048,861.

By using appropriate starting materials, Compound F can be synthesized using procedures similar to those described for the synthesis of Compound E.

### Example 4

### Synthesis of Compound G (4-11a)

### 3(S or R)-(2-Methyl-pyrimidin-5-yl)-5-oxo-9-(5,6,7,8-tetrahydro-[1,8]naphthyridin-2-yl)-nonanoic acid (4-11a)

### Step A: 6-Oxo-heptanoic acid methyl ester (4-2)

To a rapidly stirred mixture of diethyl ether (175 ml) and 40% KOH (52 ml) at 0° C. was added MNNG (15.4 g, 105 mmol). The mixture was stirred for 10 minutes. The ethereal layer was transferred to a solution of 6-oxo-heptanoic acid 4-1 (5.0 g, 34.68 mmol) and CH₂Cl₂ at 0° C. The solution was purged with argon for 30 minutes and then concentrated. Flash chromatography (silica, 30% to 50% EtOAc/hexanes) gave ester 4-2 as a clear oil.

TLC R_{f}=0.88 (silica, EtOAc).

¹H NMR (300 MHz, CDCl₃) δ 3.67 (s, 3H), 2.46 (m, 2H), 2.33 (m, 2H), 2.14 (s, 3H), 1.62 (m, 4H).

### Step B: 5-[1,8]-Naphthyridin-2-yl-pentanoic acid methyl ester (4-4)

A mixture of 4-2 (1.4 g, 9.04 mmol), 1-3, 2-amino-3-formylpyridine (552 mg, 4.52 mmol) (for preparation, see: J. Org. Chem., 1983, 48, 3401), and proline (260 mg, 2.26 mmol) in absolute ethanol (23 mL) was heated at reflux for 18 h. Following evaporative removal of the solvent, the residue was chromatographed (silica gel, 80% ethyl acetate/hexane, then ethyl acetate) to give ester 4-4 as a white solid.

TLC R_{f}=0.38 (silica, EtOAc).

¹H NMR (300 MHz, CDCl₃) δ 9.08 (m, 1H), 8.16 (d, J=8.0 Hz, 1H), 8.10 (d, J=8.3 Hz, 1H), 7.45 (m, 1H), 7.39 (d, J=8.3 Hz, 1H), 3.66 (s, 3H), 3.08 (t, J=7.6 Hz, 2H), 2.39 (t, J=7.6 Hz, 2H), 1.94 (m, 2H), 1.78 (m, 2H).

### Step C: 5-(5,6,7,8-Tetrahydro-[1,8]naphthyridin-2-yl)-pentanoic acid methyl ester (4-5)

A mixture of 4-4 (630 mg, 2.58 mmol) and 10% Pd/carbon (95 mg) in EtOH (25 mL) was stirred under a balloon of hydrogen for 72 h. Following filtration and evaporative removal of the solvent, the residue was chromatographed (silica gel, 70% ethyl acetate/hexanes) to give 4-5 as a colorless oil.

TLC R_{f}=0.58 (silica, ethyl acetate).

¹H NMR (300 MHz, CDCl₃) δ 7.05 (d, J=7.3 Hz, 1H), 6.34 (d, J=7.3 Hz, 1H), 4.72 (s, 1H), 3.66 (s, 3H), 3.40 (m, 2H), 2.69 (t, J=6.3 Hz, 2H), 2.53 (m, 2H), 2.33 (m, 2H), 1.90 (m, 2H), 1.66 (m, 4H).

### Step D: 2-Oxo-6-(5,6,7,8-tetrahydro-[1,8]-naphthyridin-2-yl)-hexyl-phosphonic acid dimethyl ester (4-6)

A solution of dimethyl methylphosphonate (13.20 g, 106.5 mmol) in anhydrous THF (165 mL) was cooled to −78° C. and treated dropwise with 2.5 M n-BuLi (42.3 mL). After stirring at −78° C. for 45 min, a solution of ester 4-5 (6.6 g, 26.6 mmol) in THF (35 mL) was added dropwise and the resulting solution stirred for 30 min at −78° C., quenched with sat. NH₄Cl (100 mL), then extracted with ethyl acetate (3×150 mL). The combined organic extracts were dried (MgSO₄), filtered, and concentrated to afford a yellow oil. Chromatography on silica gel (5% MeOH/CH₂Cl₂) afforded 4-6 as a yellow oil.

Rf (silica, 5% MeOH/CH₂Cl₂)=0.20.

1H NMR (300 MHz, CDCl₃) δ 7.05 (d, J=7.3 Hz, 1H), 6.34 (d, J=7.32 Hz, 1H), 4.80 (br, s, 1H), 3.81 (s, 3H), 3.75 (s, 3H), 3.4 (m, 2H), 3.08 (d, J=22.7 Hz), 2.7-2.5 (m, 6H), 1.91 (m, 2H), 1.68 (m, 4H).

### Step E: 1-(2-Methyl-pyrimidin-5-yl)-7-(5,6,7,8-tetrahydro-[1,8]naphthyridin-2-yl)-hept-1-en-3-one (4-7)

To a solution of 4-6 (5.5 g, 16.2 mmol), 5-formyl-2-methylpyrimidine (4-6a, 1.8 g, 14.7 mmol; for preparation, see J. Heterocyclic Chem., 28, 1281 (1991)) in 40 mL DMF was added K₂CO₃ (4.07 g, 32 mmol). The mixture was stirred at ambient temperature for 15 hr, and concentrated to a paste. The residue was diluted with water, extracted with ethyl acetate, and dried over magnesium sulfate. Following concentration, the residue was chromatographed on silica gel (70 chloroform/25 ethyl acetate/5 methanol) to give 4-7 as a white solid.

R_{f}=0.20 (silica, 70 chloroform/20 ethyl acetate/10 methanol).

¹H NMR (400 MHz, CDCl₃) δ 8.80 (s, 2H), 7.44 (d, 1H, J=16 Hz), 7.05 (d, 1H, J=7 Hz), 6.81 (d, 1H, J=16 Hz), 6.35 (d, 1H, J=7 Hz), 4.72 (br s, 1H), 3.39 (m, 2H), 2.69 (s, 3H), 2.64 (m, 4H), 2.58 (m, 2H), 1.91 (m, 2H), 1.74 (m, 4H).

### Step F: 2-[1(S or R)-(2-Methyl-pyrimidin-5-yl)-3-oxo-7-(5,6,7,8-tetrahydro-[1,8]naphthyridin-2-yl)-heptyl]-malonic acid diethyl ester (4-8a)

To a solution of 4-7 (1.0 g, 2.97 mmol) and diethyl malonate (0.717 ml, 4.5 mmol) in ethanol (20 mL) and THF (20 mL) was added sodium ethoxide (0.1 mL of a 30% w/w solution in ethanol). After 4 hr, the mixture (4-8) was concentrated, and the residue purified on a 5×50 cm Chiralcel AD column (flow=80 mL/min, A:B=30:70) (A=0.1% diethylamine/hexane, B=2-propanol). Product 4-8a eluted at 15 minutes; its enantiomer, 4-8b eluted at 26 minutes.

¹H NMR (400 MHz, CDCl₃) δ 8.53 (s, 2H), 7.02 (d, 1H, J=7 Hz), 6.28 (d, 1H, J=7 Hz), 4.07 (br s, 1H), 4.18 (m, 2H), 4.02 (m, 2H), 3.92 (m, 1H), 3.72 (m, 2H), 3.39 (m, 2H), 2.94 (m, 2H), 2.64 (s, 3H), 2.42 (m, 2H), 2.33 (m, 2H), 1.89 (m, 2H), 1.60 (m, 4H), 1.26 (m, 4H), 1.19 (t, 3H, J=3 Hz).

### Step G: 3(S or R)-(2-Methyl-pyrimidin-5-yl)-5-oxo-9-(5,6,7,8-tetrahydro-[1,8]naphthyridin-2-yl)-nonanoic acid ethyl ester (4-10a)

To a solution of 4-8a (0.530 g, 1.07 mmol) in ethanol (5 mL) was added NaOH (1.12 mL of 1N solution in water, 1.12 mmol). After stirring at 40° C. for 30 minutes, the mixture was treated with HCl (1.12 mL of 1N solution in water, 1.12 mmol) and concentrated. The residue was suspended in toluene (20 mL) and heated at reflux. After 1 h, evaporation of the solvents gave 4-10a as a yellow oil.

R_{f}=0.32 (silica, 70 chloroform/20 ethyl acetate/10 methanol).

¹H NMR (400 MHz, CDCl₃) δ 8.54 (s, 2H), 7.04 (d, 1H, J=7 Hz), 6.31 (d, 1H, J=7 Hz), 4.86 (br s, 1H), 4.04 (q, 2H, J=3 Hz), 3.63 (m, 1H), 3.40 (m, 2H), 2.94-2.48 (m, 9H), 2.37 (m, 4H), 1.89 (m, 2H), 1.57 (m, 4H), 1.19 (t, 3H, J=3 Hz).

### Step H: 3(S or R)-(2-Methyl-pyrimidin-5-yl)-5-oxo-9-(5,6,7,8-tetrahydro-[1,8]naphthyridin-2-yl)-nonanoic acid (4-11a)

To a solution of 4-10a (0.15 g, 0.353 mmol) in ethanol (1 mL) was added NaOH (0.39 mL of 1N solution in water, 0.39 mmol). After 30 minutes, the mixture was concentrated, and the residue chromatographed on silica gel (20:10:1:1 to 10:10:1:1 ethyl acetate/ethanol/NH₄OH/water) to give 4-11a as a white solid.

R_{f}=0.21 (silica, 10:10:1:1 ethyl acetate/ethanol/NH₄OH/water).

¹H NMR (400 MHz, CH₃OD) δ 8.62 (s, 2H), 7.43 (d, 1H, J=7 Hz), 3.68 (m, 1H), 3.43 (m, 2H), 3.02 (m, 2H), 2.80 (m, 3H), 2.59 (m, 10H), 1.91 (m, 2H), 1.60 (m, 3H).

### Example 5

### Synthesis of Compound H (5-11)

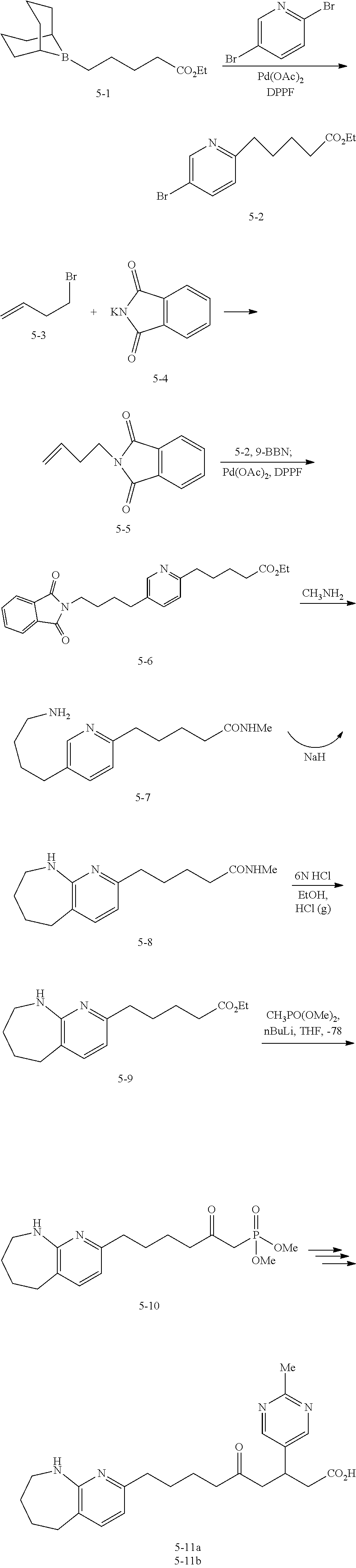

### 3(R) and 3(S)-(2-Methyl-pyrimidin-5-yl)-5-oxo-9-(5,6,7,8-tetrahydro-5H-pyrido[2,3-b]azepin-2-yl)-nonanoic acid (5-11a and 5-11b)

### Step A: 5-(5-Bromo-pyridin-2-yl)-pentanoic acid ethyl ester (5-2)

To a stirred solution of ethyl-1-pentenoic acid (10 g, 78 mmol) in degassed THF (80 mL) at 0° C. was added dropwise a solution of 9-BBN (187 mL of 0.5 M in THF, 94 mmol) and the mixture stirred for 18 hours at ambient temperature to produce 5-1. K₂CO₃ (18.4 g, 133 mmol) and 2,5-dibromopyridine (18.5 g, 78 mmol) were added, followed by a premixed and aged (70° C. for 30 min) suspension of Pd(OAc)₂ (2.0 g, 8.9 mmol) and DPPF (5.4 g, 9.8 mmol) in degassed DMF (80 mL). The resulting mixture was stirred for 18 hours at 70° C., cooled, diluted with ethyl acetate, washed with water and brine, dried over MgSO₄, and concentrated. To the stirring residue dissolved in THF (400 mL) was added water (150 mL) and NaHCO₃ (33 g) and after 10 minutes, NaBO₃.H₂O (48 g). After vigorous stirring for 30 minutes, the mixture was diluted with ethyl acetate, washed with water and brine, dried over MgSO₄, and concentrated to an oil. The residue was chromatographed on silica gel (10-20% EtOAc/hexane) to give 5-2 as a colorless oil.

TLC R_{f}=0.75 (silica, 40% EtOAc/hexane).

¹H NMR (400 MHz, CDCl₃): δ 8.57 (s, 1H), 7.70 (m, 1H), 7.05 (d, 1H, J=8 Hz), 4.15 (q, 2H, J=6 Hz), 2.77 (t, 2H, J=7 Hz), 2.34 (t, 2H, J=7 Hz), 1.7 (m, 4H), 1.26 (t, 3H, J=6 Hz).

### Step B: 2-But-3-enyl-isoindole-1,3-dione (5-5)

To a stirred solution of 4-bromo-1-butene (5-3, 20 g, 148 mmol) in DMF (150 mL) was added potassium phthalimide (5-4, 25 g, 133 mmol) and the mixture stirred for 18 hours at 70° C. After cooling to RT, the mixture was diluted with ether, washed with water and brine, dried over MgSO₄, and concentrated to give 5-5 as a white solid.

¹H NMR (400 MHz, CDCl₃): δ 7.85 (m, 2H), 7.72 (m, 2H), 5.82 (m, 1H), 5.08 (m, 2H), 3.77 (t, 2H, J=7 Hz), 2.44 (m, 2H).

### Step C: 5-{5-[4-(1,3-Dioxo-1,3-dihydro-isoindol-2-yl)-butyl]-pyridin-2-yl}-pentanoic acid ethyl ester (5-6)

To a stirred solution of 5-5 (4.23 g, 21 mmol) in degassed THF (20 mL) at 0° C. was added dropwise a solution of 9-BBN (50.4 mL of 0.5 M in THF, 25.2 mmol) and the mixture stirred for 18 hours at ambient temperature. K₂CO₃ (5.0 g, 35.8 mmol) and 5-2 (5.0 g, 17.4 mmol) were added, followed by a premixed and aged (70° C. for 30 min) suspension of Pd(OAc)₂ (0.54 g, 2.4 mmol) and DPPF (1.45 g, 2.6 mmol) in degassed DMF (20 mL). The resulting mixture was stirred for 18 hours at 70° C., cooled, diluted with ethyl acetate, washed with water and brine, dried over MgSO₄, and concentrated. To the stirring residue dissolved in THF (200 mL) was added water (75 mL) and NaHCO₃ (16.5 g) and after 10 minutes, NaBO₃.H₂O (24 g). After vigorous stirring for 30 minutes, the mixture was diluted with ethyl acetate, washed with water and brine, dried over MgSO₄, and concentrated to an oil. The residue was chromatographed on silica gel (20-40% EtOAc/hexane) to give 5-6 as a yellow solid.

TLC R_{f}=0.31 (silica, 50% EtOAc/hexane).

¹H NMR (400 MHz, CDCl₃): δ 8.37 (s, 1H), 7.84 (m, 2H), 7.75 (m, 2H), 7.40 (m, 1H), 7.05 (m, 1H), 4.12 (q, 2H, J=7 Hz), 3.71 (m, 2H), 2.78 (t, 2H, J=7 Hz), 2.61 (t, 2H, J=7 Hz), 2.33 (t, 2H, J=7 Hz), 1.64 (m, 8H), 1.23 (t, 3H, J=6 Hz).

### Step D: 5-[5-(4-Amino-butyl)-pyridin-2-yl]-pentanoic acid methylamide (5-7)

A mixture of 5-6 (45 g, 110 mmol) and a saturated solution of methylamine in methanol (300 mL) in a sealed tube was heated at 70° C. for 12 hours. The mixture was cooled and concentrated to an oil. The residue was chromatographed on silica gel (10:10:1:1 EtOAc/EtOH/NH₄OH/H₂O) to give 5-7 as a yellow oil.

TLC R_{f}=0.16 (silica, 10:10:1:1 EtOAc/EtOH/NH₄OH/H₂O).

¹H NMR (400 MHz, CDCl₃): δ 8.32 (s, 1H), 7.41 (m, 1H), 7.07 (m, 1H), 2.74 (m, 7H), 2.59 (t, 2H, J=6 Hz), 2.21 (t, 2H, J=6 Hz), 1.69 (m, 6H), 1.48 (m, 2H).

### Step E: 5-(6,7,8,9-Tetrahydro-5H-pyrido[2,3-b]azepin-2-yl)-pentanoic acid methylamide (5-8)

A mixture of 5-7 (24 g, 91.2 mmol) and NaH (10.9 g of a 60% weight dispersion in mineral oil, 273 mmol) in xylenes (500 mL) was purged with argon for 30 min, and then heated at reflux for 72 hours. The mixture was cooled, quenched with ethanol, diluted with 10% aqueous potassium carbonate and extracted with ethyl acetate. The organics were dried over MgSO₄, and concentrated to an oil. The residue was chromatographed on silica gel (70:25:5 CHCl₃/EtOAc/MeOH/H₂O) to give 5-8 as a white solid.

TLC R_{f}=0.15 (silica, 70:25:5 CHCl₃/EtOAc/MeOH).

¹H NMR (400 MHz, CDCl₃): δ 7.24 (d, 1H, J=7 Hz), 6.53 (d, 1H, J=7 Hz), 5.43 (br s, 1H), 4.62 (br s, 1H), 3.12 (m, 2H), 2.79 (d, 3H, J=5 Hz), 2.63 (m, 4H), 2.18 (m, 2H), 1.81 (m, 2H), 1.68 (m, 6 Hz).

### Step F: 5-(6,7,8,9-Tetrahydro-5H-pyrido[2,3-b]azepin-2-yl)-pentanoic acid ethyl ester (5-9)

A mixture of 5-8 (3 g, 11.5 mmol) and 6 M HCl (100 mL) in a sealed tube was heated at 70° C. for 12 hours. The mixture was cooled and concentrated to an oil. The residue was azeotroped from ethanol (50 mL) twice, then dissolved in 4 M HCl in ethanol (100 mL) and heated at 70° C. for 1 hour. The mixture was cooled and concentrated to an oil. The residue was diluted with ethyl acetate, washed with 10% aqueous potassium carbonate and brine, dried over MgSO₄, and concentrated to give 5-9 as a brown oil.

TLC R_{f}=0.44 (silica, 70:25:5 CHCl₃/EtOAc/MeOH).

¹H NMR (400 MHz, CDCl₃): δ 7.22 (d, 1H, J=7 Hz), 6.53 (d, 1H, J=7 Hz), 4.63 (br s, 1H), 4.11 (q, 2H, J=7 Hz), 3.12 (m, 2H), 2.66 (m, 2H), 2.62 (t, 2H, J=6 Hz), 2.33 (t, 2H, J=6 Hz), 1.70 (m, 2H), 1.63 (m, 6H), 1.27 (t, 3H, J=7 Hz).

### Step G: 3(R) and 3(S)-(2-Methyl-pyrimidin-5-yl)-5-oxo-9-(6,7,8,9-tetrahydro-5H-pyrido[2,3-b]azepin-2-yl)-nonanoic acid (5-11a and 5-11b)

Utilizing the procedures for the conversion of 5-5 into 5-11a and 5-11b, 5-9 was converted into 5-11a and 5-11 b by way of 5-10. Resolution of the enantiomers was carried out by chiral chromatography of the keto diester intermediate corresponding to 5-8 on a Chiralcel AD column (10 cm×50 cm) using 70% A/30% B (A=2-propanol; B=0.1% diethylamine in hexanes) at a flow rate of 250 mL/min.

TLC R_{f}=0.21 (silica, 10:10:1:1 EtOAc/EtOH/NH₄OH/H₂O).

¹H NMR (400 MHz, CDCl₃): δ 8.63 (s, 2H), 7.42 (d, 1H, J=7 Hz), 6.55 (d, 1H, J=7 Hz), 3.64 (m, 1H), 3.31 (m, 2H), 3.05 (m, 1H), 2.87 (m, 1H), 2.77 (m, 2H), 2.58 (m, 9H), 1.84 (m, 4H), 1.57 (m, 4H).

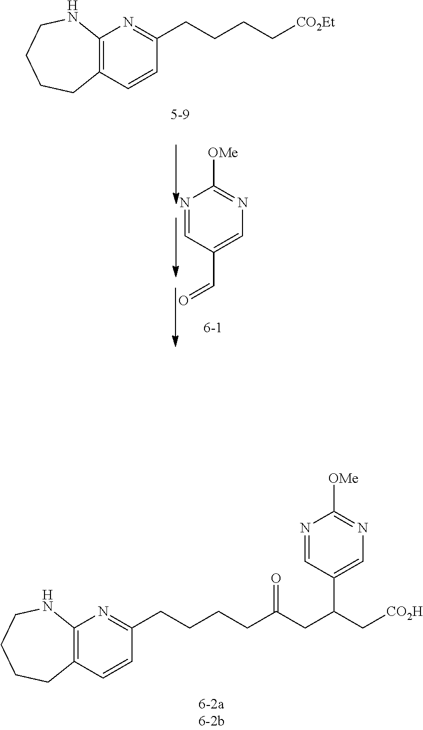 [SMILES: CCOC(=O)CCCCc1ccc2c(n1)NCCCC2 ; COc1ncc(C=O)cn1 ; COc1ncc(C(CC(=O)O)CC(=O)CCCCc2ccc3c(n2)NCCCC3)cn1]

### 3(R) and 3(S)-(2-Methoxy-pyrimidin-5-yl)-5-oxo-9-(5,6,7,8-tetrahydro-5H-pyrido[2,3-b]azepin-2-yl)-nonanoic acid (6-2a and 6-2b)

Utilizing the procedures for the conversion of 5-5 into 5-11a and 5-11b, 5-9 and 2-methoxy-pyrimidine-5-carbaldehyde (6-1, for preparation, see J. Heterocycl. Chem. (1991), 28, 1281) were converted into 6-2a and 6-2b. Resolution of the enantiomers was carried out by chiral chromatography of the keto diester intermediate corresponding to 1-8 on a Chiralcel AD column (10 cm×50 cm) using 70% A/30% B (A=2-propanol; B=0.1% diethylamine in hexanes) at a flow rate of 250 mL/min.

TLC R_{f}=0.21 (silica, 10:10:1:1 EtOAc/EtOH/NH₄OH/H₂O).

¹H NMR (400 MHz, CDCl₃): δ 8.48 (s, 2H), 7.42 (d, 1H, J=7 Hz), 6.56 (d, 1H, J=7 Hz), 3.94 (s, 3H), 3.62 (m, 1H), 3.29 (m, 2H), 2.98 (m, 1H), 2.85 (m, 1H), 2.79 (m, 2H), 2.58 (m, 2H), 1.84 (m, 4H), 1.57 (m, 4H).

### ASSAYS

### SPAV3 Assay

### Materials:

1. Wheat germ agglutinin Scintillation Proximity Beads (SPA): Amersham
2. Octylglucopyranoside: Calbiochem
3. HEPES: Calbiochem
4. NaCl: Fisher
5. CaCl₂: Fisher
6. MgCl₂: SIGMA
7. Phenylmethylsulfonylfluoride (PMSF): SIGMA
8. Optiplate: PACKARD
9. (S)-3-(4-(2-(6-aminopyridin-2-yl)ethyl)benzamido)-2-((4-(iodo-¹²⁵I)phenyl)sulfonamido)propanoic acid as found in WO0046215 (specific activity 500-1000 Ci/mmole)
10. Test compound
11. Purified integrin receptor: αvβ3 was purified from 293 cells overexpressing αvβ3 (Duong et al., J. Bone Min. Res., 8:S378, 1993) according to Pytela (Methods in Enzymology, 144:475, 1987)
12. Binding buffer: 50 mM HEPES, pH 7.8, 100 mM NaCl, 1 mM Ca²⁺/Mg²⁺, 0.5 mM PMSF
13. 50 mM octylglucoside in binding buffer: 50-OG buffer

### Procedure:

1. Pretreatment of SPA beads:
   500 mg of lyophilized SPA beads were first washed four times with 200 ml of 50-OG buffer and once with 100 ml of binding buffer, and then resuspended in 12.5 ml of binding buffer.
2. Preparation of SPA beads and receptor mixture
   In each assay tube, 2.5 μl (40 mg/ml) of pretreated beads were suspended in 97.5 μl of binding buffer and 20 ml of 50-OG buffer. 5 ml (˜30 ng/μl) of purified receptor was added to the beads in suspension with stirring at room temperature for 30 minutes. The mixture was then centrifuged at 2,500 rpm in a Beckman GPR Benchtop centrifuge for 10 minutes at 4° C. The pellets were then resuspended in 50 μl of binding buffer and 25 μl of 50-OG buffer.
3. Reaction
   The following were sequentially added into Optiplate in corresponding wells:
   (i) Receptor/beads mixture (75 μl)
   (ii) 25 μl of each of the following: compound to be tested, binding buffer for total binding or A-8 for non-specific binding (final concentration 1 μM)
   (iii) (S)-3-(4-(2-(6-aminopyridin-2-yl)ethyl)benzamido)-2-((4-(iodo-¹²⁵I)phenyl)sulfonamido)propanoic acid as found in WO0046215 (specific activity 500-1000 Ci/mmole) in binding buffer (25 μl, final concentration 40 pM)
   (iv) Binding buffer (125 μl)
   (v) Each plate was sealed with plate sealer from PACKARD and incubated overnight with rocking at 4° C.
4. Plates were counted using PACKARD TOPCOUNT
5. % inhibition was calculated as follows:
   A=total counts
   B=nonspecific counts
   C=sample counts

% inhibition=[{(A−B)−(C−B)}/(A−B)]/(A−B)×100

### SPAV3 Binding Assay

| Cmpd | A | B | C | D | E | F | G | H |
|---|---|---|---|---|---|---|---|---|
| SPAV3 | 0.1 | 0.1 | 0.1 | 0.1 | 0.1 | 0.1 | 0.1 | 0.2 |
| IC₅₀ | | | | | | | | |
| (nM) | | | | | | | | |

### In Vitro Selectivity Assay (Thermal Shift Assay)

Differential scanning fluorimetry was performed on a LightCycler 480 II, real-time PCR instrument (Roche Diagnostics). Human recombinant integrins from R&D Systems (αvβ1, αvβ3 and α5β1) were reconstituted at a concentration of 10 mM in assay buffer (20 mM HEPES, pH=7.3, 100 mM NaCl, 1 mM MgCl₂, 1 mM MnCl₂) and diluted in assay buffer with Sypro orange (SIGMA) to a final concentration of 400 nM integrin and 5× Sypro orange. A volume of 4.9 μL of this mixture of protein and dye was transferred to a 384-well plate and 100 nL of DMSO or Compound A, dissolved in DMSO, were added using an Echo 555 instrument (Labcyte). The final concentration of Compound A in the assay was 20 μM. After mixing, the assay plate was sealed, spun at 1,000×g for two minutes, and subsequently heated from 25 to 99° C. over the course of 31 min in the LightCycler 480 II instrument. Fluorescence intensity was measured using excitation/emission wavelengths of 465 and 580 nm, respectively. Changes in protein thermal stability (ΔTₘ) upon compound binding were analyzed by using LightCycler 480 (software provided by the manufacturer).

### Solid Phase Receptor Assay

The assay was performed according to the method described in International Patent Publication WO 2014/015054 A1 “Beta Amino Acid Derivatives As Integrin Antagonists.” Briefly, 96-well plates were coated overnight with purified fibronectin or vitronectin (R&D Systems) in TBS+ buffer (25 mM Tris 7.4, 137 mM NaCl, 2.7 mM KCl, 1 mM CaCl₂, 1 mM MgCl₂, 1 mM MnCl₂). Compound A was added at different concentrations to recombinant human integrin proteins (R&D Systems) reconstituted in TBS+/0.1% bovine serum albumin, and 50 μL of this mixture were added to the empty wells of the coated plate and incubated for 1-2 hours. After 3 washes, 50 μL of biotinylated antibody (R&D Systems) in TBS+/0.1% bovine serum albumin were added. The procedure continued with three more washes, addition of 50 μL of streptavidin-conjugated horseradish peroxidase (R&D Systems), and incubation for 20 minutes. After 3 more washes, 50 μL of tetramethylbenzidine (TMB) substrate (SIGMA) were added and plates were read after 20 min by colorimetric detection at 650 nm wavelength in an EnVision Multilabel Plate Reader (Perkin Elmer). The concentrations of fibronectin used were: 2 μg/mL for the α5β1 assay and 5 μg/mL for the αvβ1 assay. The concentrations of vitronectin used were: 1 μg/mL for the αvβ3 assay and 0.25 μg/mL for the αvβ5 assay. The biotinlylated antibodies used were: biotinlylated anti-αv antibody for αvβ1, αvβ3 and αvβ5, and biotinlylated anti-α5 antibody for α5β1.

### The Effects of Compound A in the Solid Phase and Thermal Shift Assays.

| | Solid Phase Assay | Thermal Shift Assay |
|---|---|---|
| | Compound A | ΔTm (° C.) at |
| | IC₅₀(nM) | 20 μM Compound A |
| αvβ1 | 2.1/0.9 | 19.4/15.6 |
| αvβ3 | 5.2/0.33 | 21.5/16.4 |
| α5β1 | 4.9/19.6 | 8.1/14.0 |

IC₅₀ and ΔTm values were obtained from two different experiments

### In Vitro Podocyte Assays

The effects of Compound A on podocyte motility were evaluated using Oris cell migration assay kit in 96-well plates (coated with either vitronectin or fibronectin). The Oris™ Cell Migration Assay is designed with a unique cell seeding stopper or biocompatible gel, detection mask, and stopper tool. These unique plate designs generate highly reproducible results using a microscope, digital imaging system. 7 to 10-day differentiated human podocytes (100 ul of 50,000 cells/ml) were seeded in each well of the ORIS plate. After sitting at room temperature for about 15 minutes, the plate was placed into 37° C. incubator and podocytes were incubated with complete podocyte medium for 24 h. Stoppers from each well (keeping stoppers in 4 wells which were served as time zero control) were carefully removed, medium discarded, and fresh (10% FBS) podocyte medium added to each well. Podocytes were pretreated with Compound A (10 uM to 0.01 nM) for 2 h prior to puromycin animonucleoside (PAN, 30 ug/ml or 15 ug/ml) treatment. Podocytes were then treated with PAN in the presence or absence of Compound A at different concentrations (ranging from 10 uM to 0.01 nM) in 10% FBS medium for 48 h. Podocytes were then fixed with 4% paraformaldehyde in PBS for 30 minutes (adding 50 ul to each well). After discarding the fixative, podocytes were stained with Hoechst 33342 (stock 10 at working concentration at 5 uM) for 30 min. Podocytes were then washed with PBS three times. Finally, after adding 100 ul of PBS to each well, plate was sealed with a black cover and kept at 4° C. until image analysis. Images of podocyte motility were captured using Acumen eX3 (manufacturer TTP Labtech Ltd. address: Melbourn Science Park, Melbourn, Hertfordshire SG8 6EE, United Kingdom). Compound A significantly inhibited human podocyte motility response induced by puromycin, in dose-dependent manner, in vitronectin or fibronectin coated plates (Table 1, 2, 3, 4) with an IC₅₀ of 9.94 nM in vitronectin coated plates or an IC₅₀ of 1.12 nM in fibronectin coated plates.

### Effects of Compound A (“A”) on Puromycin (PAN)-Induced Human Podocyte Motility on Vitronectin (VN) Coated-96-Well Plate

| | | | PAN + | PAN + | | PAN + | | | | | | |
|---|---|---|---|---|---|---|---|---|---|---|---|---|
| | | | A (10 | A | PAN + A | A | PAN + A | PAN + A | PAN + A | PAN + A | PAN + A | PAN + A |
| Groups | Veh. | PAN | uM) | (1 uM) | (100 nM) | (10 nM) | (3.16 nM) | (1 nM) | (0.316 nM) | (0.1 nM) | (0.0316 nM) | (0.01 nM) |
| % | 40.5 ± | 43.6 ± | 16.9 ± | 16.1 ± | 19.6 ± | 28.6 ± | 31.3 ± | 42.1 ± | 36.6 ± 2.2* | 43.0 ± 2.6 | 44.0 ± 2.2 | 55.1 ± 4.4* |
| migrated | 0.7 | 1.6 | 3.6*** | 1.8*** | 2.2*** | 4.0** | 1.3*** | 2.5 | | | | |
| cells vs | | | | | | | | | | | | |
| control | | | | | | | | | | | | |
| cells | | | | | | | | | | | | |

Data are expressed as Mean ± SEM.
PAN: puromycin (30 ug/ml),
***p < 0.001 vs PAN,
**p < 0.01 vs PAN,
*p < 0.05 vs PAN

Effect of Compound A (“A”) on human podocyte motility was examined using Oris cell migration assay in vitronectin (VN) coated-96-well plate. Puromycin (PAN, 30 ug/ml) treated podocytes showed slightly higher motility compared to untreated vehicle (Veh.) groups. Compound A treatment in podocytes for 48 hours significantly inhibited podocyte motility, in a dose-dependent manner, compared to PAN-treated group, with an IC₅₀ of 9.94 nM.

### Effects of Compound A (“A”) Alone on Human Podocyte Motility on Vitronectin (VN) Coated-96-Well Plate

| | | A (10 | A (1 | A | A | | | A | A | A | A |
|---|---|---|---|---|---|---|---|---|---|---|---|
| Groups | Veh. | uM) | uM) | (100 nM) | (10 nM) | A (3.16 nM) | A (1 nM) | (0.316 nM) | (0.1 nM) | (0.0316 nM) | (0.01 nM) |
| % | 34.7 ± 0.6 | 17.9 ± | 17.9 ± | 20.5 ± | 19.2 ± 1.5*** | 30.6 ± 0.8** | 33.4 ± 1.6 | 38.6 ± 2.8 | 38.8 ± 1.4 | 43.7 ± 2.7* | 39.7 ± 1.4 |
| migrated | | 1.8*** | 2.2*** | 1.0*** | | | | | | | |
| cells vs | | | | | | | | | | | |
| control | | | | | | | | | | | |
| cells | | | | | | | | | | | |

Data are expressed as Mean ± SEM. Veh: Vehicle
***p < 0.001 vs Veh.,
**p < 0.01 vs Veh.,
*p < 0.05 vs Veh. (One-way ANOVA followed by T-tests)

Effect of Compound A (“A”) alone on human podocyte motility was examined using Oris cell migration assay in vitronectin (VN) coated-96-well plate. Compared to vehicle (Veh.) treated group, Compound A treatment in podocytes for 48 hours showed significant inhibition of motility in a dose-dependent manner.

### Effects of Compound A (“A”) on Puromycin (PAN)-Induced Human Podocyte Motility on Fibronectin (FN) Coated-96-Well Plate

| | | | PAN + | PAN + | PAN + | PAN + | | | | | | |
|---|---|---|---|---|---|---|---|---|---|---|---|---|
| | | | A | A (1 | A | A | PAN + A | PAN + | PAN + A | PAN + A | PAN + A | PAN + A |
| Groups | Veh. | PAN | (10 uM) | uM) | (100 nM) | (10 nM) | (3.16 nM) | A (1 nM) | (0.316 nM) | (0.1 nM) | (0.0316 nM) | (0.01 nM) |
| % | 79.4 ± | 86.9 ± | 33.1 ± | 36.3 ± | 35.8 ± | 54.1 ± | 64.8 ± | 72.9 ± 3.3* | 83.6 ± 3.6 | 84.9 ± 3.5 | 80.3 ± 4.0 | 76.3 ± 7.2 |
| migrated | 2.5 | 3.7 | 4.1*** | 3.4*** | 3.7*** | 4.7*** | 6.2** | | | | | |
| cells vs | | | | | | | | | | | | |
| control | | | | | | | | | | | | |
| cells | | | | | | | | | | | | |

Data are expressed as Mean ± SEM. PAN: puromycin (15 ug/ml).
***p < 0.001 vs PAN,
**p < 0.01 vs PAN,
*p < 0.05 vs PAN (One-way ANOVA followed by T-tests)

Effect of Compound A (“A”) on human podocyte motility was examined using Oris cell migration assay in fibronectin (FN) coated-96-well plate. Puromycin (PAN, 15 ug/ml) treated podocytes slightly increased podocyte motility compared to untreated vehicle (Veh.) groups. Compound A treatment in podocytes for 48 hours significantly inhibited podocyte motility, in a dose-dependent manner, with an IC₅₀ of 1.12 nM.

### Effects of αvβ3 Antagonist on Renal Function, Plasma Triglycerides, Plasma Cholesterol, Kidney Collagen I, Kidney Collagen III, Renal Histology, Glomerular Filtration Rate, Fibrosis Score, and mRNA Expression in ZSF1 Rats

The effects of Compound A (“A”) on renal function, plasma triglycerides, plasma cholesterol, kidney collagen I, kidney collagen III, renal histology, glomerular filtration rate, fibrosis score, and mRNA gene expression (profibrotic genes and integrin β3) were evaluated in male obese ZSF1 rats (a hybrid F1 of Zucker diabetic fatty rat and spontaneously hypertensive heart failure rat; a diabetic nephropathy model) when administered as in-feed for 28 weeks. Sixty obese male ZSF1 rats were randomized to five groups: Obese control (n=12), Compound A 60 mpk (n=12), Compound A 200 mpk (n=12), Compound A 400 mpk (n=12), Enalapril 10 mpk (n=12); Eight lean male ZSF1 rats were used for normal control. Renal functional changes were monitored by blood and urine analysis following in-feed dosing for 28 weeks. Compound exposure was also monitored during the study.

Upon completion of the study, animals were euthanized and blood and organs (kidney, heart, aorta, eyes, and lumber vertebrae (LV1-LV5) and left femur) were collected for histology assessment (including EM for the kidneys) or DEXA scan (lumber vertebrae and left femur). Kidney tissues were fixed in 10% formalin and then paraffin embedded. Tissue sections were stained with hematoxylin and eosin (H&E), periodic acid-Schiff (PAS), and Masson's trichrome and evaluated under light microscope. The severity of histopathologic changes in renal tubules, interstitium, vasculature, and glomeruli were graded on a 1 to 5 scale corresponding to minimal, mild, moderate, marked, and severe as described previously [21, 22]. Sections from both kidneys were examined. Collagen deposition in the kidney was graded on a 1 to 5 scale corresponding to minimal, mild, moderate, marked, and severe, based on the blue stained area size and intensity.

Following deparaffinization and rehydration, each kidney tissue section was processed to identify collagen I and III deposition. The primary antibodies used were rabbit anti-type I collagen polyclonal antibody (Abcam, Cambridge, Mass.) diluted at 2 ug/ml, and rabbit anti-type III collagen polyclonal antibody (Lifespan, Seattle, Wash.) at 3 ug/ml. The signal was developed by using Super PicTure HRP Polymer Rabbit Primary kit (Invitrogen) and the slides were counterstained with hematoxylin. The Aperio ScanScope XT Slide Scanner (Aperio Technologies, Vista, Calif.) system was used to capture whole slide digital images with a 20× objective. Digital images were managed using Aperio Spectrum. The positive stains were identified and quantified using a macro created from a color deconvolution algorithm (Aperio Technologies, Vista, Calif.).

As shown in the tables below, Compound A (“A”) had no significant effect on body weight (BW), food intake (FI) and water intake (WI).

### Effects of Compound A (“A”) on Body Weight (Grams).

| | | Obese | Obese A | Obese A | Obese A | Obese |
|---|---|---|---|---|---|---|
| Treatment | Lean control | vehicle | 60 mpk | 200 mpk | 400 mpk | Enalapril |
| weeks | (n = 8) | (n = 12) | (n = 12) | (n = 12) | (n = 12) | 10 mpk (n = 12) |
| −2 | 386.9 ± 8.7 | 522.2 ± 7.6 | 521.3 ± 6.9 | 520.6 ± 6.4 | 522.3 ± 7.1 | 522.2 ± 10.2 |
| 1 | 430.8 ± 11.9 | 568.3 ± 7.2 | 566.6 ± 7.3 | 568.6 ± 7.4 | 568.3 ± 6.5 | 570.2 ± 10.3 |
| 2 | 445.3 ± 12.3 | 579.8 ± 8.0 | 577.4 ± 7.2 | 581.5 ± 7.0 | 582.8 ± 6.5 | 578.3 ± 11.2 |
| 4 | 459.8 ± 13.0 | 600.8 ± 8.3 | 599.8 ± 7.4 | 605.9 ± 7.1 | 604.1 ± 7.2 | 585.7 ± 11.1 |
| 6 | 483.0 ± 11.4 | 635.0 ± 9.7 | 624.7 ± 9.0 | 631.8 ± 7.7 | 631.8 ± 8.9 | 606.1 ± 11.6 |
| 8 | 502.1 ± 10.6 | 600.8 ± 8.3 | 652.0 ± 9.4 | 663.0 ± 7.4 | 654.5 ± 10.6 | 623.8 ± 13.3 |
| 10 | 520.0 ± 10.4 | 682.7 ± 13.2 | 678.0 ± 10.9 | 690.6 ± 8.5 | 676.5 ± 12.7 | 637.2 ± 14.1 |
| 12 | 534.9 ± 11.2 | 705.7 ± 14.5 | 703.7 ± 11.9 | 718.9 ± 9.3 | 704.8 ± 12.8 | 655.6 ± 14.2* |
| 14 | 545.8 ± 11.3 | 714.7 ± 16.8 | 712.1 ± 12.7 | 729.3 ± 11.9 | 704.4 ± 14.6 | 658.1 ± 16.2** |
| 16 | 556.6 ± 12.2 | 735.3 ± 17.0 | 733.8 ± 13.2 | 754.1 ± 11.9 | 739.3 ± 15.0 | 671.1 ± 16.8** |
| 18 | 570.3 ± 12.6 | 753.2 ± 17.5 | 746.9 ± 14.1 | 772.3 ± 13.1 | 757.9 ± 16.9 | 682.1 ± 16.4** |
| 21 | 582.3 ± 13.3 | 780.6 ± 19.8 | 781.1 ± 14.1 | 800.2 ± 13.9 | 791.2 ± 18.4 | 709.5 ± 18.0** |
| 24 | 597.5 ± 13.4 | 809.0 ± 19.5 | 808.9 ± 14.6 | 825.7 ± 14.7 | 823.8 ± 18.9 | 733.7 ± 20.2** |
| 28 | 606.0 ± 15.9 | 836.1 ± 18.3 | 839.0 ± 14.9 | 848.1 ± 16.4 | 852.1 ± 19.5 | 753.7 ± 21.8** |

*p < 0.05,
**p < 0.01,
Enalapril vs. obese vehicle (Two-way ANOVA followed by Tukey)

### Effects of Compound A (“A”) on Food Intake (Grams/24 h).

| | Lean | Obese | Obese A | Obese A | Obese A | Obese |
|---|---|---|---|---|---|---|
| Treatment | control | vehicle | 60 mpk | 200 mpk | 400 mpk | Enalapril |
| weeks | (n = 8) | (n = 12) | (n = 12) | (n = 12) | (n = 12) | 10 mpk (n = 12) |
| −2 | 22.2 ± 0.6 | 37.9 ± 1.4 | 38.6 ± 1.0 | 37.7 ± 1.4 | 37.7 ± 1.1 | 37.5 ± 1.5 |
| 1 | 19.4 ± 0.6 | 28.3 ± 1.5 | 30.5 ± 1.2 | 29.0 ± 1.0 | 30.0 ± 1.6 | 30.1 ± 1.3 |
| 2 | 21.2 ± 0.5 | 30.0 ± 1.7 | 26.9 ± 1.0 | 29.2 ± 1.1 | 32.0 ± 1.2 | 31.7 ± 1.4 |
| 4 | 20.4 ± 0.7 | 32.9 ± 2.0 | 29.5 ± 2.1 | 30.6 ± 1.0 | 33.6 ± 0.9 | 33.1 ± 1.0 |
| 6 | 19.8 ± 1.1 | 33.5 ± 1.6 | 32.8 ± 0.9 | 34.3 ± 1.6 | 34.6 ± 1.2 | 33.1 ± 1.0 |
| 8 | 18.0 ± 1.5 | 36.0 ± 1.4 | 34.1 ± 1.1 | 31.8 ± 1.2 | 34.2 ± 1.4 | 35.1 ± 1.5 |
| 12 | 22.4 ± 1.0 | 38.0 ± 1.3 | 36.7 ± 1.6 | 35.6 ± 0.9 | 35.7 ± 0.7 | 36.6 ± 1.5 |
| 16 | 20.3 ± 0.8 | 35.9 ± 1.1 | 33.2 ± 1.3 | 33.6 ± 1.5 | 33.6 ± 0.7 | 33.3 ± 1.6 |
| 21 | 22.5 ± 0.5 | 36.2 ± 1.0 | 34.6 ± 1.6 | 35.6 ± 0.9 | 36.0 ± 1.2 | 37.7 ± 1.6 |
| 24 | 19.1 ± 0.8 | 32.2 ± 0.9 | 31.2 ± 1.0 | 31.7 ± 1.2 | 33.3 ± 1.5 | 35.5 ± 1.3 |
| 28 | 18.9 ± 0.8 | 32.9 ± 0.9 | 31.6 ± 1.0 | 33.3 ± 1.2 | 32.1 ± 1.7 | 33.6 ± 1.5 |

Effects of Compound A (“A”) on Water Intake (mls/24 h)

| | Lean | Obese | Obese A | Obese A | Obese A | Obese |
|---|---|---|---|---|---|---|
| Treatment | control | vehicle | 60 mpk | 200 mpk | 400 mpk | Enalapril |
| weeks | (n = 8) | (n = 12) | (n = 12) | (n = 12) | (n = 12) | 10 mpk (n = 12) |
| −2 | 29.2 ± 1.6 | 62.6 ± 6.4 | 60.8 ± 4.5 | 59.1 ± 5.9 | 57.2 ± 4.9 | 68.1 ± 4.9 |
| 1 | 25.2 ± 0.5 | 36.8 ± 5.4 | 34.6 ± 3.4 | 30.7 ± 1.9 | 37.6 ± 2.8 | 50.2 ± 6.2 |
| 2 | 27.1 ± 0.7 | 43.6 ± 7.1 | 34.5 ± 2.2 | 29.2 ± 2.5 | 40.3 ± 3.1 | 55.0 ± 5.2 |
| 4 | 28.1 ± 0.9 | 45.1 ± 6.9 | 46.0 ± 5.7 | 33.4 ± 2.0 | 45.0 ± 3.7 | 56.6 ± 6.0 |
| 6 | 27.5 ± 1.0 | 52.4 ± 8.0 | 45.8 ± 4.5 | 39.4 ± 2.8 | 49.9 ± 4.8 | 61.7 ± 5.5 |
| 8 | 24.8 ± 1.6 | 64.8 ± 7.1 | 59.2 ± 4.5 | 51.4 ± 4.6 | 62.2 ± 5.6 | 85.4 ± 7.4* |
| 12 | 29.8 ± 1.4 | 71.8 ± 6.8 | 71.5 ± 4.9 | 63.9 ± 4.8 | 63.5 ± 5.3 | 91.7 ± 7.5 |
| 16 | 30.7 ± 1.5 | 66.4 ± 6.5 | 63.7 ± 4.9 | 57.3 ± 5.0 | 63.0 ± 3.9 | 83.3 ± 8.3 |
| 21 | 30.3 ± 0.9 | 59.1 ± 4.0 | 64.5 ± 8.5 | 58.1 ± 3.6 | 59.3 ± 4.4 | 75.8 ± 5.9 |
| 24 | 25.2 ± 0.9 | 57.8 ± 5.0 | 59.4 ± 4.7 | 61.8 ± 3.5 | 67.2 ± 4.8 | 89.7 ± 7.9** |
| 28 | 25.8 ± 1.3 | 62.0 ± 3.9 | 66.5 ± 6.6 | 70.5 ± 5.6 | 80.1 ± 4.6 | 68.0 ± 6.5 |

*p < 0.05,
**p < 0.01,
Enalapril vs. obese vehicle (Two-way ANOVA followed by Tukey)

As shown in the tables below, Compound A (“A”) at 400 mpk significantly decreased urinary protein/creatinine ratio (UPCR) at 16-, 21-, 24- and 28-week of treatment time point.
Compound A (“A”) at 400 mpk significantly decreased 24 h urinary protein excretion at 16-, 24- and 28-week of treatment time point.
Effects of Compound A (“A”) on UPCR (m/m).

| | Lean | Obese | Obese A | Obese A | Obese A | Obese |
|---|---|---|---|---|---|---|
| Treatment | control | vehicle | 60 mpk | 200 mpk | 400 mpk | Enalapril |
| weeks | (n = 8) | (n = 12) | (n = 12) | (n = 12) | (n = 12) | 10 mpk (n = 12) |
| −2 | 1.7 ± 0.1 | 10.8 ± 0.8 | 10.9 ± 0.9 | 10.7 ± 0.9 | 10.8 ± 0.9 | 10.8 ± 0.8 |
| 1 | 1.1 ± 0.1 | 11.2 ± 0.8 | 12.3 ± 1.0 | 10.7 ± 0.8 | 12.3 ± 0.9 | 6.3 ± 0.3 |
| 2 | 1.1 ± 0.1 | 11.7 ± 1.1 | 11.9 ± 1.0 | 10.9 ± 1.0 | 12.5 ± 0.9 | 7.4 ± 0.4 |
| 4 | 1.2 ± 0.1 | 15.1 ± 1.4 | 13.4 ± 1.1 | 12.1 ± 1.1 | 12.7 ± 0.6 | 8.0 ± 0.4** |
| 6 | 0.9 ± 0.1 | 15.7 ± 1.4 | 16.6 ± 1.2 | 14.1 ± 1.2 | 13.8 ± 0.9 | 7.3 ± 0.4** |
| 8 | 0.8 ± 0.0 | 21.1 ± 1.8 | 20.2 ± 1.4 | 18.0 ± 1.5 | 17.8 ± 1.2 | 10.2 ± 0.6** |
| 12 | 0.9 ± 0.1 | 28.5 ± 1.9 | 30.8 ± 1.7 | 28.7 ± 1.6 | 26.9 ± 1.5 | 17.1 ± 0.9** |
| 16 | 0.7 ± 0.1 | 30.8 ± 1.9 | 27.3 ± 1.7 | 26.3 ± 1.5 | 20.9 ± 1.0++ | 14.0 ± 0.7** |
| 21 | 0.8 ± 0.1 | 33.5 ± 2.4 | 27.7 ± 2.0+ | 28.1 ± 1.6+ | 26.0 ± 1.3++ | 15.6 ± 0.6** |
| 24 | 0.7 ± 0.1 | 36.2 ± 2.0 | 34.4 ± 2.3 | 32.0 ± 1.9 | 28.1 ± 1.7++ | 16.6 ± 0.8** |
| 28 | 1.0 ± 0.2 | 41.0 ± 3.2 | 37.5 ± 2.8 | 36.5 ± 2.3 | 31.2 ± 1.7++ | 19.5 ± 0.8** |

**p < 0.01 Enalapril 10 mpk vs. obese vehicle.
+P < 0.05, Compound A (“A”) 60 mpk vs. obese vehicle;
++p < 0.01, A 400 mpk vs. obese vehicle (Two-way ANOVA followed by Tukey)

Effects of Compound A (“A”) on 24 h Urinary Protein Excretion (mg/24 h).

| | Lean | Obese | Obese A | Obese A | | Obese |
|---|---|---|---|---|---|---|
| Treatment | control | vehicle | 60 mpk | 200 mpk | Obese A | Enalapril |
| weeks | (n = 8) | (n = 12) | (n = 12) | (n = 12) | 400 mpk (n = 12) | 10 mpk (n = 12) |
| −2 | 19.3 ± 1.3 | 127.9 ± 11.3 | 128.8 ± 11.0 | 122.3 ± 9.5 | 127.0 ± 11.0 | 132.9 ± 9.9 |
| 1 | 15.7 ± 1.6 | 121.2 ± 11.4 | 137.2 ± 12.7 | 117.7 ± 9.4 | 138.5 ± 11.3 | 73.9 ± 3.8 |
| 2 | 17.0 ± 1.4 | 137.9 ± 15.5 | 139.6 ± 11.8 | 124.9 ± 10.9 | 148.8 ± 12.3 | 91.2 ± 4.9 |
| 4 | 18.2 ± 1.0 | 174.8 ± 20.0 | 158.4 ± 13.7 | 140.0 ± 12.4 | 148.0 ± 8.3 | 96.1 ± 5.1 |
| 6 | 14.5 ± 1.2 | 205.1 ± 21.9 | 197.9 ± 15.7 | 167.1 ± 15.0 | 162.6 ± 11.7 | 92.2 ± 6.0** |
| 8 | 13.4 ± 0.8 | 296.1 ± 30.3 | 265.7 ± 18.3 | 233.9 ± 18.7 | 224.8 ± 16.3 | 135.3 ± 10.2** |
| 12 | 16.1 ± 1.8 | 428.6 ± 35.3 | 425.3 ± 21.6 | 400.5 ± 20.5 | 358.4 ± 19.9 | 234.8 ± 16.4** |
| 16 | 14.6 ± 1.7 | 480.0 ± 38.5 | 462.2 ± 30.4 | 452.2 ± 27.6 | 360.5 ± 19.7++ | 252.2 ± 21.1** |
| 21 | 17.4 ± 2.3 | 533.0 ± 38.8 | 486.2 ± 39.2 | 489.0 ± 28.6 | 448.1 ± 19.6 | 274.5 ± 16.8** |
| 24 | 15.2 ± 2.8 | 622.3 ± 40.6 | 582.0 ± 43.9 | 541.6 ± 32.3 | 483.8 ± 27.1++ | 289.4 ± 16.5** |
| 28 | 22.1 ± 4.4 | 711.3 ± 53.4 | 654.3 ± 48.1 | 646.2 ± 44.6 | 554.9 ± 28.3++ | 309.6 ± 15.8** |

**p < 0.01, Enalapril 10 mpk vs. obese vehicle;
++p < 0.01, Compound A (“A”) 400 mpk vs. obese vehicle. (Two-way ANOVA followed by Tukey)

### GFR Measurement by FITC-Sinistrin Clearance

For the measurement of GFR, a miniaturized device (NIC-Kidney, Mannheim Pharma & Diagnostics, Mannheim, Germany) was used. In brief, the device (batteries, diodes, and microprocessor) containing an optical component was affixed on a depilated region of the back using a double-sided sticky patch (Lohmann GmbH KG, 56567, Neuwied, Germany) under isofluorane anesthesia (3% isoflurane mixed with oxygen). After a resting baseline period of 1-1.5 minutes, a bolus of FITC-sinistrin (5 mg/100 g body weight, dissolved in 0.5 mL sterile isotonic saline) was injected through the tail vein. The rat was then placed in a clean cage for recovery from anesthesia to responsible ambulation. The conscious rat was observed for the next 2 hours during the data collection via the miniaturized device. The excretion kinetics of FITC-sinistrin was determined using a sampling rate of 60 measurements per minute with an excitation time of 10 milliseconds per measurement for 120 minutes after the injection. One compartment model was used for FITC-sinistrin clearance [18]. After completion of GFR measurement, the device was gently removed from the skin and the rat returned to its home cage.

As shown in the table below, Compound A (“A”) at 200 mpk or 400 mpk at week 28 showed improvement of renal function as measured by FITC-sinistrin clearance (expressed as % change of improvement when compared to Obese Vehicle group).

| | | | | Obese |
|---|---|---|---|---|
| Treat- | Obese | Obese A | Obese A | Enalapril |
| ment | vehicle | 200 mpk | 400 mpk | 10 mpk |
| weeks | (n = 12) | (n = 12) | (n = 12) | (n = 12) |
| 28 | 0 | 8.8% | 14.4% | 59.6%**** |

****p < 0.0001, Enalapril (at 10 mpk) vs. obese vehicle (One-way ANOVA)

As shown in the table below, Compound A (“A”) at 60 mpk, 200 mpk and 400 mpk at weeks 28-30 had no significant effect on glomerular filtration rate.
Effects of Compound A (“A”) on Glomerular Filtration Rate (uLs/Min/100 gm BW).

| | Lean | Obese | Obese A | Obese A | Obese A | Obese |
|---|---|---|---|---|---|---|
| Treatment | control | vehicle | 60 mpk | 200 mpk | 400 mpk | Enalapril |
| weeks | (n = 8) | (n = 12) | (n = 12) | (n = 12) | (n = 12) | 10 mpk (n = 12) |
| 28 | 1102 ± 43.5 | 608.6 ± 41.0 | 609.8 ± 42.1 | 663.2 ± 50.6 | 697.5 ± 37.9 | 971.9 ± 53.3**** |

****p < 0.0001, Enalapril vs. obese vehicle (One-way ANOVA followed by Tukey)

As shown in the table below, Compound A (“A”) 200 mpk at week 4 and 400 mpk at week 16 and 28 significantly decreased plamsa triglyceride levels.
Effects of Compound A (“A”) on Plamsa Triglyceride (mg/dl).

| | | | | | | Obese |
|---|---|---|---|---|---|---|
| Treatment | Lean control | Obese vehicle | Obese A | Obese A | Obese A | Enalapril |
| weeks | (n = 8) | (n = 12) | 60 mpk (n = 12) | 200 mpk (n = 12) | 400 mpk (n = 12) | 10 mpk (n = 12) |
| −2 | 121.1 ± 3.8 | 2201.4 ± 173.0 | 2046.8 ± 72.0 | 1965.3 ± 83.6 | 2211.5 ± 117.2 | 2023.9 ± 82.1 |
| 2 | 99.9 ± 10.6 | 2571.6 ± 222.3 | 2535.3 ± 170.6 | 2111.1 ± 123.5 | 2415.9 ± 119.5 | 2979.8 ± 161.7 |
| 4 | 99.3 ± 8.1 | 3499.4 ± 323.7 | 2807.5 ± 143.4 | 2592.4 ± 206.8* | 2763.1 ± 146.9 | 3212.4 ± 137.6 |
| 6 | 97.1 ± 15.0 | 3195.1 ± 237.5 | 2873.2 ± 116.5 | 2534.6 ± 185.5 | 2715.6 ± 111.4 | 2866.6 ± 145.5 |
| 8 | 120.0 ± 15.1 | 3612.4 ± 193.0 | 3187.9 ± 157.1 | 3325.3 ± 189.8 | 3185.0 ± 191.8 | 3770.3 ± 280.6 |
| 12 | 131.8 ± 16.1 | 3478.4 ± 234.9 | 3138.5 ± 168.9 | 3188.3 ± 191.4 | 3086.5 ± 121.3 | 3298.3 ± 221.4 |
| 16 | 127.9 ± 17.8 | 3464.8 ± 278.6 | 3120.3 ± 191.6 | 2930.4 ± 217.5 | 2648.9 ± 167.8* | 3554.6 ± 186.3 |
| 21 | 175.3 ± 20.1 | 3173.3 ± 298.6 | 2933.4 ± 317.5 | 2717.2 ± 199.8 | 2523.9 ± 220.7 | 3224.3 ± 240.5 |
| 24 | 157.1 ± 16.3 | 3143.8 ± 282.8 | 3093.3 ± 355.2 | 2923.8 ± 267.3 | 2439.5 ± 162.4 | 3180.2 ± 204.5 |
| 28 | 217.9 ± 14.5 | 3149.2 ± 255.8 | 3218.7 ± 427.5 | 2632.8 ± 269.6 | 2215.7 ± 179.3* | 2855.1 ± 291.3 |

*p < 0.05, Compound A 200 mpk (at w 4) and 400 mpk (at w16 and w 28) vs. obese vehicle. (Two-way ANOVA followed by Tukey)

As shown in the table below, Compound A (“A”) 200 mpk at week 28 and 400 mpk at week 16, 21, 24, and 28 significantly decreased plasma cholesterol levels.

| | | Obese | Obese A | | | Obese |
|---|---|---|---|---|---|---|
| Treatment | Lean control | vehicle | 60 mpk | Obese A | Obese A | Enalapril |
| weeks | (n = 8) | (n = 12) | (n = 12) | 200 mpk (n = 12) | 400 mpk (n = 12) | 10 mpk (n = 12) |
| −2 | 81.0 ± 1.5 | 197.6 ± 8.4 | 192.0 ± 4.5 | 189.5 ± 6.6 | 195.5 ± 5.5 | 187.1 ± 5.4 |
| 2 | 88.1 ± 1.7 | 216.4 ± 7.4 | 229.3 ± 9.6 | 205.4 ± 7.8 | 229.8 ± 6.0 | 220.9 ± 5.9 |
| 4 | 86.8 ± 1.5 | 276.6 ± 11.9 | 262.7 ± 10.5 | 243.5 ± 9.2 | 252.3 ± 5.3 | 251.1 ± 7.4 |
| 6 | 73.9 ± 2.3 | 266.4 ± 12.6 | 261.2 ± 9.9 | 233.7 ± 9.1 | 238.9 ± 6.6 | 232.5 ± 6.0 |
| 8 | 86.5 ± 2.4 | 302.8 ± 11.6 | 289.1 ± 9.5 | 289.3 ± 9.3 | 278.8 ± 9.5 | 294.3 ± 6.7 |
| 12 | 90.9 ± 2.2 | 321.8 ± 12.1 | 315.4 ± 13.2 | 315.9 ± 11.2 | 306.4 ± 7.0 | 290.2 ± 7.6 |
| 16 | 93.5 ± 2.9 | 379.2 ± 19.7 | 357.8 ± 13.3 | 346.8 ± 12.1 | 304.6 ± 11.1** | 319.3 ± 9.9* |
| 21 | 102.4 ± 2.7 | 407.1 ± 21.6 | 392.9 ± 20.6 | 366.7 ± 14.1 | 351.4 ± 13.8* | 321.1 ± 11.3** |
| 24 | 107.1 ± 2.2 | 431.1 ± 38.6 | 448.0 ± 23.8 | 415.1 ± 19.6 | 374.7 ± 12.8* | 354.3 ± 12.5** |
| 28 | 115.9 ± 2.6 | 527.2 ± 30.1 | 495.7 ± 29.4 | 451.9 ± 23.6** | 391.2 ± 14.7** | 368.8 ± 13.2** |

*p < 0.05
**p < 0.01,
Enalapril 10 mpk or Compound A at 200 mpk or 400 mpk vs. obese vehicle. (Two-way ANOVA followed by Tukey)

As shown in the table below, Compound A (“A”) at 400 mpk at week 28 significantly decreased kidney collagen I protein levels (expressed as % of area) when compared to Obese Vehicle group.

| | | | | Obese |
|---|---|---|---|---|
| Treat- | Obese | Obese A | Obese A | Enalapril |
| ment | vehicle | 200 mpk | 400 mpk | 10 mpk |
| weeks | (n = 12) | (n = 12) | (n = 12) | (n = 12) |
| 28 | 42.09 ± 1.42 | 39.79 ± 0.82 | 36.45 ± 0.85** | 34.83 ± 0.71*** |

**p < 0.01, ***p < 0.001, Compound A (at 400 mpk) or Enalapril (at 10 mpk) vs. obese vehicle (Two-way ANOVA followed by Tukey)

As shown in the table below, Compound A (“A”) 400 mpk at week 28 significantly decreased kidney collagen III protein levels (expressed as % of area) when compared to Obese Vehicle group.

| | | | | Obese |
|---|---|---|---|---|
| Treat- | Lean | Obese | Obese A | Enalapril |
| ment | control | vehicle | 400 mpk | 10 mpk |
| weeks | (n = 8) | (n = 12) | (n = 12) | (n = 12) |
| 28 | 12.89 ± 0.72 | 22.90 ± 1.09 | 19.23 ± 0.76* | 19.19 ± 0.90* |

*p < 0.05, Compound A (at 400 mpk) or Enalapril (at 10 mpk) vs. obese vehicle (One-way ANOVA)

As shown in the table below, Compound A (“A”) at 400 mpk at week 28 significantly decreased renal fibrosis score (expressed as 0-5) when compared to Obese Vehicle group.

| | | | | Obese |
|---|---|---|---|---|
| Treat- | Obese | Obese A | Obese A | Enalapril |
| ment | vehicle | 200 mpk | 400 mpk | 10 mpk |
| weeks | (n = 12) | (n = 12) | (n = 12) | (n = 12) |
| 28 | 3.0 ± 0.14 | 2.7 ± 0.1 | 2.2 ± 0.1*** | 2.0 ± 0.1*** |

***p < 0.001, Compound A (at 400 mpk) or Enalapril (at 10 mpk) vs. obese vehicle (One-way ANOVA followed by proportional odds logistic regression)

As shown in the table below, Compound A (“A”) at 200 mpk or 400 mpk at week 28 significantly decreased expression of key profibrotic genes and integrin beta3 in the kidney when compared to obese vehicle group.

| | | | | | Obese |
|---|---|---|---|---|---|
| | Lean | Obese | Obese A | Obese A | Enalapril |
| | control | vehicle | 200 mpk | 400 mpk | 10 mpk |
| | (n = 8) | (n = 12) | (n = 12) | (n = 12) | (n = 12) |
| PAI-1 | 0.23 ± 0.01 | 1.02 ± 0.06 | 0.80 ± 0.03 | 0.66 ± 0.03** | 0.52 ± 0.04**** |
| Collagen I (a1) | 0.28 ± 0.02 | 1.02 ± 0.06 | 0.79 ± 0.04 | 0.66 ± 0.02** | 0.54 ± 0.02*** |
| Collagen III (a1) | 0.22 ± 0.02 | 1.05 ± 0.10 | 0.60 ± 0.03**** | 0.52 ± 0.01**** | 0.39 ± 0.02**** |
| Integrin beta3 | 0.57 ± 0.03 | 1.01 ± 0.04 | 0.83 ± 0.03* | 0.74 ± 0.03** | 0.68 ± 0.03**** |

Data is expressed as mean ± SEM.
*P < 0.05,
**P < 0.01,
***P < 0.001,
****P < 0.0001,
One-Way ANOVA followed by Dunnett's multiple comparison with Obese Vehicle.

What is claimed is:

1. A method for treating a disease selected from diabetic nephropathy, focal segmental glomerulosclerosis, nephrotic syndrome, non-diabetic chronic kidney disease, renal fibrosis or acute kidney injury with an RGD mimetic integrin receptor antagonist.

2. The method of claim 1 wherein the RGD mimetic integrin receptor antagonist is selected from: 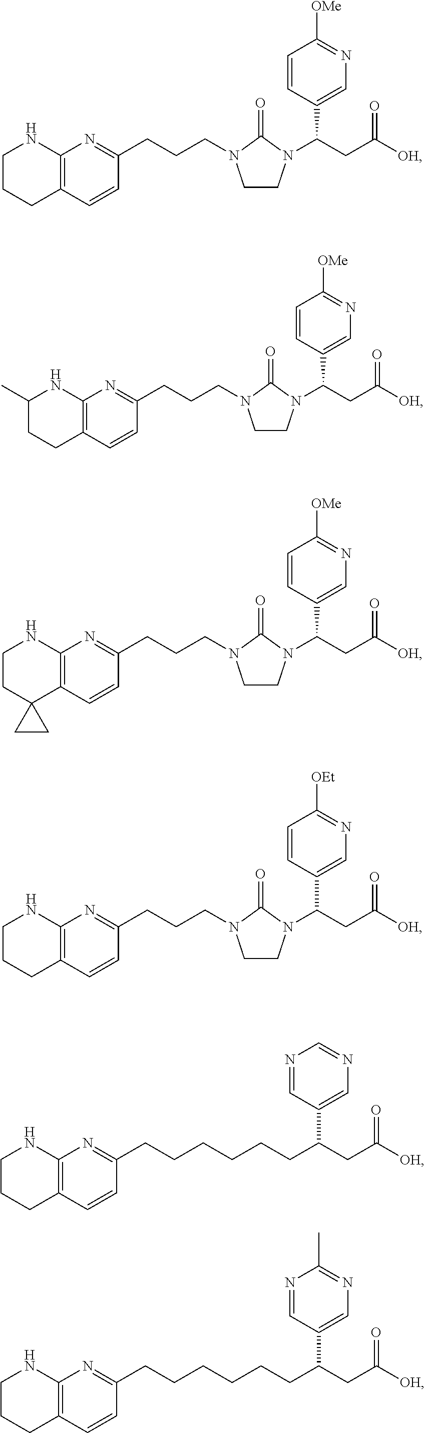 [SMILES: COc1ccc([C@H](CC(=O)O)N2CCN(CCCc3ccc4c(n3)NCCC4)C2=O)cn1 ; COc1ccc([C@H](CC(=O)O)N2CCN(CCCc3ccc4c(n3)NC(C)CC4)C2=O)cn1 ; COc1ccc([C@H](CC(=O)O)N2CCN(CCCc3ccc4c(n3)NCCC43CC3)C2=O)cn1 ; CCOc1ccc([C@H](CC(=O)O)N2CCN(CCCc3ccc4c(n3)NCCC4)C2=O)cn1 ; O=C(O)C[C@H](CCCCCCc1ccc2c(n1)NCCC2)c1cncnc1 ; Cc1ncc([C@@H](CCCCCCc2ccc3c(n2)NCCC3)CC(=O)O)cn1] 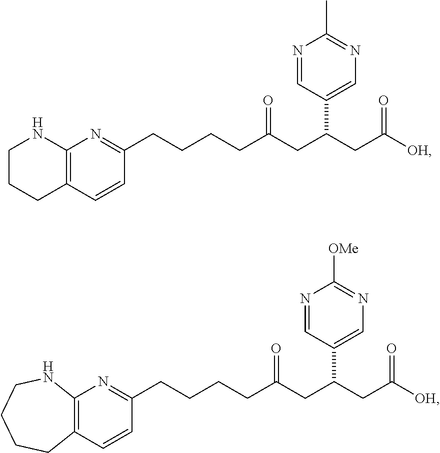 [SMILES: Cc1ncc([C@H](CC(=O)O)CC(=O)CCCCc2ccc3c(n2)NCCC3)cn1 ; COc1ncc([C@H](CC(=O)O)CC(=O)CCCCc2ccc3c(n2)NCCCC3)cn1]
or a pharmaceutically acceptable salt thereof.

3. The method of claim 2 wherein the RGD mimetic integrin receptor antagonist is 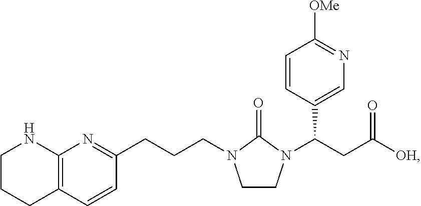 [SMILES: COc1ccc([C@H](CC(=O)O)N2CCN(CCCc3ccc4c(n3)NCCC4)C2=O)cn1]
or a pharmaceutically acceptable salt thereof.

4. The method of claim 1 wherein the disease is diabetic nephropathy.

5. The method of claim 1 further comprising an additional agent selected from an anti-hypertensive agent, anti-atherosclerotic agent, anti-diabetic agent and/or anti-obesity agent.

6. The method of claim 5 wherein the additional agent is selected from an angiotensin converting enzyme inhibitor; dual inhibitor of angiotensin converting enzyme (ACE) and neutral endopeptidase (NEP); angiotensin II receptor antagonist; a thiazide-like diuretic; potassium sparing diuretic; carbonic anhydrase inhibitor; neutral endopeptidase inhibitor; aldosterone antagonist; aldosterone synthase inhibitor; renin inhibitor; endothelin receptor antagonist; vasodilator; calcium channel blocker; potassium channel activator; sympatholitics; beta-adrenergic blocking drug; alpha adrenergic blocking drug; nitrate; nitric oxide donating compound; lipid lowering agent; a cholesterol absorption inhibitor; niacin; niacin receptor agonist; niacin receptor partial agonist; metabolic altering agent; alpha glucosidase inhibitor; dipeptidyl peptidase inhibitor; ergot alkaloids; phosphodiesterase-5 (PDE5) inhibitor; or a combination thereof.

7. The method of claim 6 wherein the additional agent is enalapril.

8. The method of claim 6 wherein the additional agent is losartan.

9. The method of claim 6 wherein the additional agents are enalapril and losartan.

10. The method of claim 3 further comprising enalapril.

11. The method of claim 3 further comprising losartan.
